(19) United States
(12) Patent Application Publication
(10) Pub. No.: US 2016/0229777 A1
(43) Pub. Date: Aug. 11, 2016

(54) PURIFICATION OF AN ACETYL STREAM

(71) Applicant: Eastman Chemical Company, Kingsport, TN (US)

(72) Inventors: Scott Donald Barnicki, Kingsport, TN (US); Daniel McNabb, Kingsport, TN (US); Jerry Lynn Miller, Kingsport, TN (US); Randy Lynn Jennings, Gate City, VA (US)

(73) Assignee: EASTMAN CHEMICAL COMPANY, Kingsport, TN (US)

(21) Appl. No.: 14/618,449

(22) Filed: Feb. 10, 2015

(51) Int. Cl. C07C 45/48; B01D 3/36

(52) U.S. Cl. CPC C07C 45/48; B01D 3/36

(57) ABSTRACT

A process for purifying an acetyl stream containing an impurity that forms an azeotrope with the acetyl moiety includes the steps of a) contacting an acetyl feed stream comprising: i) acetic acid; and ii) an acetic acid azeotrope-forming compound, with a metal oxide catalyst in a ketonization reactor to produce a crude product mixture from a ketonization reaction comprising acetone, water, the acetic acid azeotrope-forming compound, and byproducts from the ketonization reaction; and b) distilling the crude product mixture to recover: i) a lower boiling fraction stream comprising acetone, and a minor amount of: water, said acetic acid azeotrope-forming compound, and ketonization byproducts, and ii) a higher boiling fraction stream comprising a major amount of: water, the acetic acid azeotrope forming compound, and the ketonization byproducts. Optionally, the process can include contacting the crude product mixture with water in a countercurrent absorber prior to the distilling step (b).

### BACKGROUND OF THE INVENTION

1. Field of the Invention

The present invention relates to preparing acetone from an acetyl stream containing an impurity that forms an azeotrope with the acetyl moiety. More particularly, the present invention relates to purifying an acetyl stream wherein the acetyl moiety is converted to acetone which is then readily separated from the acetyl-azeotrope forming impurity.

2. Background of the Invention

It is known to those skilled in the art that numerous industrial processes are presently used to manufacture acetone. One process for producing acetone includes dehydrogenation of 2-propanol. Propylene is absorbed in concentrated sulfuric acid to produce isopropyl sulfate, which is then hydrolyzed to 2-propanol. The 2-propanol is then oxidized to produce acetone. It is also known that alumina-supported platinum or rhodium catalysts can be used for dehydrogenating lower secondary alcohols to ketones.

Acetone may also be produced by reacting formaldehyde with methyl chloride to produce acetone and hydrogen chloride. Methyl chloride is a toxic gas however, and formaldehyde is a known carcinogen.

Another method more commonly used is obtaining acetone as a co-product of phenol production where benzene is alkylated in the presence of a catalyst with propylene to produce cumene. Cumene is in turn oxidized to cumene hydroperoxide (CHP), which is then hydrolyzed in an acidic medium to yield phenol and acetone. Crude acetone resulting from the production of phenol from cumene typically contains about 200-700 ppm aldehydes and 200-500 ppm methanol. Traditionally, removal of light aldehyde impurities is accomplished by reactive distillation in which an aqueous solution of sodium hydroxide is injected into the distillation column to promote condensation of aldehydes to form higher-boiling compounds. Treatment of acetone with aqueous sodium hydroxide during distillation leads to production of distillation bottoms containing large amounts of polymers and salts, thereby decreasing the efficiency of conventional reboilers. Moreover, base-catalyzed self-condensation of acetone reduces the yield of purified acetone. Impurity levels in commercial acetone purified by this method are still about 30-50 ppm for acetaldehyde and about 200-300 ppm for methanol.

Acetic acid and acetic anhydride are frequently used as solvents, to prepare acetate esters and to prepare other, high-boiling anhydrides. Some examples of chemical processes that produce an acetyl byproduct stream include, but are not limited to, acetylation of wood, acetylation of alcohols with acetic anhydride to form esters, carbonylation of methanol and methyl acetate to form acetic acid and acetic anhydride, preparation of ketenes and diketene from acetic acid, preparation of 2,2,4,4-tetramethyl-1,3-cyclobutanediol from isobutyric anhydride via acetic anhydride exchange; Fischer-Tropsch synthesis, sugars fermentation to produce acetic acid or vinegar, producer waters from oil and gas production, polymerization reactions, such as condensation of phenyl acetate monomers to produce polyesters or polycarbonates; preparation of fine chemicals and pharmaceuticals; preparation of carboxylic acid anhydrides from their corresponding acids by exchange with acetic anhydride; and acylation reactions. Examples of fine chemical and pharmaceutical products include but are not limited, to industrial production of ibuprofen and liquid crystal polymers. Recovery and reuse of acetyl byproduct streams from these applications improves the overall acetyl efficiency, thereby greatly reducing the cost of the acetyl feedstock. A disadvantage of these acetyl byproduct streams is that the byproduct stream frequently contains a complex mixture of impurities that form azeotropes or distillation pinch points with acetic acid and cannot be easily separated without a complex and costly distillation scheme.

As used herein, the term “azeotrope” is intended to have its commonly accepted meaning as would be understood by persons having ordinary skill in the art; that is, a compound, blend or mixture having a constant boiling temperature and having a constant composition which is the same in both vapor and liquid. The relative volatility of the components of an azeotrope at the azeotropic composition is unity.

Azeotropes may be determined experimentally or by calculations based on the vapor equilibrium properties of the chemical components. These techniques are well known to persons skilled in the art such as, for example, by using group contribution methods as exemplified by the UNIFAC method. The present invention includes binary and ternary azeotropes containing acetic acid as one of the components.

The term “pinch point” as used herein, is intended to have its commonly understood meaning in the distillation arts, that is, a binary system that exhibits a region of low relative volatility where the y-x vapor-liquid equilibrium line (y=vapor mole fraction, x=liquid mole fraction) tangentially approaches the y=x line. Such systems are commonly referred to as having “pinched” vapor-liquid equilibrium or as “pinched” systems. Separation of component mixtures typically can be accomplished by distillation and are based on differences in vapor and liquid compositions. Since pinched systems show regions with increasingly small differences in vapor-liquid composition, separation by distillation may be difficult, requiring high reflux ratios and/or a large number of theoretical stages to effect any separation.

The term “azeotrope-forming impurity” as used herein means a compound that forms an azeotrope with acetic acid. The azeotrope may be high boiling (known as a maximum boiling azeotrope), wherein the boiling point at the azeotrope composition is greater than the boiling points of the pure components at a constant pressure. The azeotrope also may be low boiling (known as a minimum boiling azeotrope), wherein the boiling point at the azeotrope composition is less than the boiling points of the pure components at a constant pressure. Some examples of various compounds that form minimum-boiling azeotropes with acetic acid include, but are not limited to, aromatic compounds, such as for example, benzene, toluene, xylenes, butyl benzenes, isopropyl toluenes, phenylacetates, styrene, ethylbenzene, and the like; hydrocarbons, such as, for example, heptane, octane, various alkenes and terpenes, such as limonene, α-pinene, β-pinene, camphene, and the like; ketones such as, for example, 4-methyl-2-pentanone, isophorone, 2-butanone, mesityl oxide, 2,4-dimethyl-3-pentanone, substituted acetophenones; esters, such as phenylacetates; alkyl halides, aryl haldides, and hydroxyalkyl halides such as, for example, epichlorohydrin, 2-iodopropane, 1-iodopropane, 1,2-dichloropropane, 1-bromobutane, 2-bromobutane, isobutyl bromides, iodobutanes, iodoisobutanes, bromobenzene, chlorobenzene, and ethyl dibromide; sulfur-containing species, such as, for example dimethyl sulfoxide, tetrahydrothiophene, diethylsulfide, and diisopropyl sulfide; amines, amides, and other nitrogen-containing species, such as for example, trimethylamine, triethylamine, pyridine, methyl pyridines, dimethyl pyridines, dimethyl acetamide, and nitroethane; alcohols, such as isobutanol, 3-methylbutanol; and esters, such as, for example isobutyl formate, and isobutyl acetate.

For the purpose of discussion of the invention and claims herein, both terms “azeotrope” and “pinch point” will be designated herein as “azeotrope” and/or “azeotrope-forming” due to the difficulty in vapor-liquid distillative separations of such compositions notwithstanding the commonly understood meaning of each.

In the acetylation of wood, acetic anhydride is contacted with wood at high temperatures and pressures. The wood acetylation process produces a byproduct stream containing acetic acid, acetic anhydride, and various terpene and terpenoid impurities that are extracted from the wood during the acetylation reaction. These terpenes and terpenoid compounds form azeotropes with acetic acid.

Similarly, in the preparation of diketene from acetic acid, acetic acid is dehydrated at high temperature to form a ketene which is condensed and absorbed in diketene solvent where it further dimerizes to form diketene. The crude diketene absorbent is then distilled to produce in the overhead a purified diketene. The bottoms product, “diketene sludge,” contains acetic acid, water, acetone, and a host of impurities that form one or more azeotropes with acetic acid.

In another example, isobutyric anhydride is produced by acetyl exchange with acetic anhydride. The isobutyric anhydride is thermally cracked to form dimethylketene, which is then purified and dimerized to give 2,2,4,4-tetramethyl-1,3-butanedione. Dimerization is followed by hydrogenation to 2,2,4,4-tetramethyl-1,3-butanediol. The isobutyric acid recycled from the dimethylketene furnace to the isobutyric anhydride production unit contains a variety of impurities, such as for example, 2,4-dimethyl-1,3-pentadiene, tetramethylethylene, diisopropyl ketone, and isopropyl isopropenyl ketone, which result from the high temperature cracking process. Many of these impurities form azeotropes with acetic acid, and contaminate the acetyl stream from the isobutyric anhydride production unit. These acetic acid azeotrope-forming impurities cannot be separated from acetic acid by simple fractional distillation.

Accordingly, there is a need for a method by which the acetic acid from such byproduct streams can be utilized or converted to another product with a reduction or elimination of the acetic acid azeotrope-forming compound(s).

### SUMMARY OF THE INVENTION

It has been discovered that acetic acid by-product stream contaminated with up to about 50 weight % of at least one impurity that forms an azeotrope or pinch point with acetic acid can be converted to acetone by a ketonization process whereby the azeotrope-forming impurity can be separated from the acetone product by distillation. Briefly, the present invention is a method for preparing a ketone from an acetic acid containing stream having an impurity comprising at least one acetic acid azeotrope-forming compound, the method comprises contacting the acetyl feed stream with a metal oxide catalyst in a ketonization reactor to produce a crude product mixture by ketonization reaction wherein the crude product mixture comprises acetone, water, the impurity, and by-products from the ketonization reaction.

This invention also includes a method for preparing a ketone from an acetic acid containing stream comprising the steps of: a) contacting the acetyl feed stream comprising acetic acid and an impurity comprising at least one acetic acid azeotrope-forming compound with a meal catalyst in a ketonization reactor to produce a crude product mixture by a ketonization reaction wherein the crude product mixture comprises acetone, water, the impurity, and by-products from the ketonization reaction; and b) distilling the crude product mixture to recover: i) a lower boiling fraction comprising acetone, and a minor amount of: water, the impurity, and ketonization by-products, and ii) a higher boiling fraction comprising a major amount of: water, the impurity, and the ketonization by-products.

This invention also includes a process for preparing a ketone from an acetic acid containing stream comprises: a) contacting an acetyl feed stream comprising: i) acetic acid; and ii) an impurity comprising at least one acetic acid azeotrope-forming compound, with a metal oxide catalyst in a ketonization reactor to produce a crude product mixture from a ketonization reaction comprising acetone, water, the impurity, and byproducts from the ketonization reaction; b) contacting the crude product mixture with an absorption solvent to produce a liquid crude absorbent stream having greater than 50 mole % of the acetone in the crude product mixture and a gaseous crude absorbent stream comprising carbon dioxide and less than 50 mole % of the acetone in the crude product mixture; and c) distilling the crude liquid absorbent stream to recover: i) a lower boiling fraction comprising acetone, and a minor amount of the: water, the impurity comprising at least one acetic acid azeotrope-forming compound, and ketonization byproducts, and ii) a higher boiling fraction comprising a major amount of the: water, acetic acid azeotrope forming compound, and the ketonization byproducts.

The present invention also includes a process for preparing a ketone from an acetic acid containing stream comprising the steps of: a) vaporizing an acetyl feed stream comprising acetic acid, an impurity comprising at least one acetic acid azeotrope-forming compound, and 0-50 weight % water, optionally mixing steam with the vaporized acetyl feed stream to produce a vaporized feed mixture; b) superheating the vaporized feed mixture to produce a superheated feed mixture; c) contacting the superheated feed mixture with a catalyst in a ketonization reactor to produce a crude product mixture comprising acetone, water, the impurity, carbon dioxide, and byproducts from the ketonization reaction; d) recovering acetone from non-condensable components of the crude product mixture to produce a recovered liquid acetone stream and a gaseous off-gas stream; e) distilling the recovered liquid acetone stream to produce: i) a purified acetone stream comprising at least 95 weight % acetone, based on the total weight of the purified acetone stream, and a minor amount of: water, the impurity, and ketonization byproducts present in the recovered liquid acetone stream; ii) a waste water stream comprising a major amount of water in the recovered liquid acetone stream, and; iii) a waste organic stream comprising a major portion of: the impurity, and the ketonization byproducts present in the recovered liquid acetone stream.

These and other objects and advantages of the present invention will become more apparent to those skilled in the art in view of the following description and the accompanying drawings wherein like parts and objects in the several views have similar reference numerals. It is to be understood that the inventive concept is not to be considered limited to the constructions disclosed herein but instead by the scope of the appended claims.

### BRIEF DESCRIPTION OF THE FIGURES

FIG. 1 is a schematic diagram depicting a possible reaction network for the ketonization of acetic acid.

FIG. 2 is a block diagram of a purification train for the ketonization of a acetyl stream.

### DETAILED DESCRIPTION OF THE INVENTION

There is provided a process for preparing a ketone from an acetic acid containing stream having an impurity comprising at least one acetic acid azeotrope-forming compound, the method comprises contacting the acetyl feed stream with a metal oxide catalyst in a ketonization reactor to produce a crude product mixture by ketonization reaction wherein the crude product mixture comprises acetone, water, the impurity, and by-products from the ketonization reaction.

Surprisingly, it has been unexpectedly discovered that during the high temperature ketonization of acetic acid to acetone, the impurity comprising an acetic acid azeotrope or pinch-forming compounds in the feed acetic acid are not thermally decomposed to lighter species and do not promote fouling of the ketonization catalyst. These impurities after exposure to ketonization conditions retain similar boiling point characteristics as the original feed impurity compounds species and, as a consequence, do not interfere with a subsequent purification of the acetone product. Thus, acetyl byproduct feed streams contaminated with up to about 50 weight % of impurities in which at least one of the impurities forms an azeotrope or pinch point with the acetic acid, can be converted to acetone by a ketonization process and the impurities subsequently separated by distillation from the acetone product. As noted above, such impurities comprise at least one azeotrope-forming compound selected from the group consisting of an alkyl aromatic hydrocarbon, a ketone, an aromatic ester, an acyclic ester, a terpene, a terpenoid, an acyclic unsaturated hydrocarbon, and combinations thereof.

As used herein the term “ketonization,” is understood to mean a process in which two carboxylic acids, carboxylic acid salts, or esters are converted to a ketone, carbon dioxide, and water, at an elevated temperature. The ketonization of carboxylic acids is well-known method for the production symmetrical and unsymmetrical ketones. Generally, ketonization is intended to be synonymous with the term “ketonic decarboxylation” and refers to a process in which ketone is formed from the decarboxylative condensation of two carboxylic acid molecules. The ketonization of acetic acid with itself and other carboxylic acids, esters, or aldehydes is a valuable and high yield means to the synthesis of acetone and other methyl ketones. The ketonization of acetic acid to acetone co-produces one mole of water along with each mole of acetone produced. Moreover, although the yield of ketone from acetic acid can be over 99 mole %, small amounts of heavier organics, such as mesityl oxide, mesitylene, isophorone, methyl ethyl ketone, and methyl propyl ketone, are formed as by-products of the ketonization reaction. Thus, a crude acetone stream derived from ketonization of acetic acid will comprise acetone, water, and by-product organics.

The ketonization of acetic acid with itself and cross ketonization of acetic acid with higher carboxylic acids are well known routes for the production of acetone and higher methyl ketones. The general reactions for self- and cross-ketonization of acetic acid are:

Self-Ketonization:

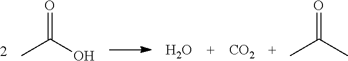

Cross-Ketonization:

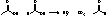

Unsymmetric methyl ketones may be produced by co-feeding other carboxylic acid with acetic acid. For example, co-feeding propionic acid with acetic acid results in the formation of methyl ethyl ketone, n-butyric acid with acetic acid results in the formation of methyl propyl ketone, isobutyric acid with acetic acid results in formation of methyl isopropyl ketone.

The product ketones, exemplified by acetone, derived via ketonization of acetic acid in a manner described above, may undergo further reaction over the ketonization catalyst, following an aldol-like condensation/dehydration pathway to form higher α,β-unsaturated ketones, most notably mesityl oxide via condensation of acetone.

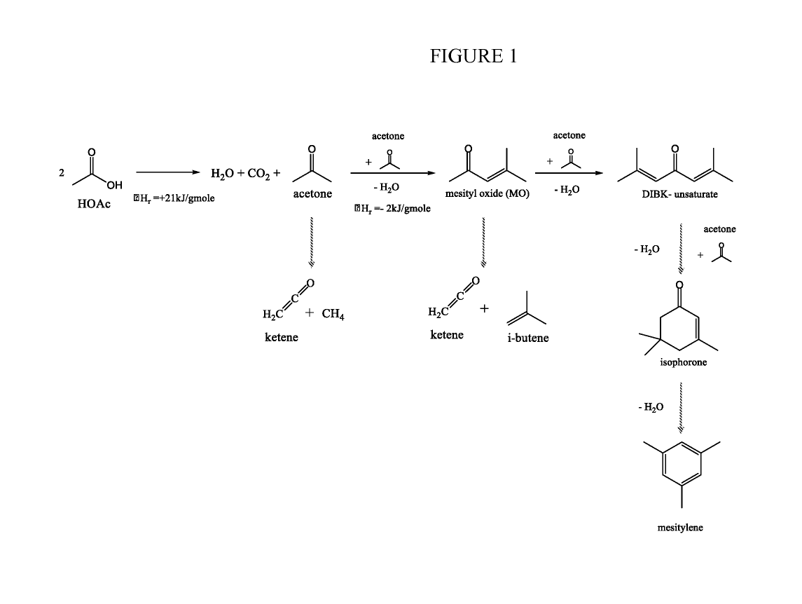

It has been found that mesityl oxide may undergo a further decomposition reaction to produce a reaction product comprising isobutylene. A more complete reaction network for the ketonization of acetic acid to acetone is shown in FIG. 1. One skilled in the art will understand cross ketonization of acetic acid with higher carboxylic acids can produce acetone and higher molecular weight methyl ketones.

The invention also includes a process for preparing a ketone from an acetic acid containing stream comprising: a) contacting an acetyl feed stream comprising acetic acid and an impurity comprising at least one acetic acid azeotrope-forming compound with a metal oxide catalyst in a ketonization reactor to produce a crude product mixture by ketonization reaction comprising acetone, water, the impurity, and by-products from the ketonization reaction; and b) distilling the crude product mixture to recover: i) a lower boiling fraction stream comprising acetone, and a minor amount of: water, the impurity, and ketonization by-products, and ii) a higher boiling fraction stream comprising a major amount of: water, the impurity, and the ketonization by-products.

The invention further includes a process for preparing a ketone from an acetic acid containing stream comprising: a) contacting an acetyl feed stream comprising: i) acetic acid; and ii) an impurity comprising at least one acetic acid azeotrope-forming compound, with a metal oxide catalyst in a ketonization reactor to produce a crude product mixture by ketonization reaction comprising acetone, water, the impurity, and byproducts from the ketonization reaction; b) contacting the crude product mixture with an absorption solvent to produce a liquid crude absorbent stream having greater than 50 mole % of the acetone in the crude product mixture and a gaseous crude absorbent stream comprising carbon dioxide and less than 50 mole % of the acetone in the crude product mixture; and c) distilling the crude liquid absorbent stream to recover: i) a lower boiling fraction stream comprising acetone, and a minor amount of the: water, the impurity comprising at least one acetic acid azeotrope-forming compound, and ketonization byproducts, and ii) a higher boiling fraction stream comprising a major amount of the: water, acetic acid azeotrope forming compound, and the ketonization byproducts.

Additionally, the invention includes a process for preparing a ketone from an acetic acid containing stream comprises the steps of: a) vaporizing an acetyl feed stream comprising acetic acid, an impurity comprising at least one acetic acid azeotrope-forming compound, and 0-50 weight % water, based on the total weight of the feed stream, optionally mixing steam with the vaporized acetyl feed stream to produce a vaporized feed mixture; b) superheating the vaporized feed mixture to produce a superheated feed mixture; c) contacting the superheated feed mixture with a metal oxide catalyst in a ketonization reactor to produce a crude product mixture by ketonization reaction comprising acetone, water, the impurity, carbon dioxide, and byproducts from the ketonization reaction; d) recovering acetone from non-condensable components of the crude product mixture to produce a liquid crude acetone stream and a gaseous off-gas stream; e) distilling the liquid crude acetone stream to recover: i) a purified acetone stream comprising at least 95 weight % acetone, based on the weight of the purified acetone stream, and a minor amount of: water, the impurity, and ketonization byproducts present in the recovered liquid acetone stream; ii) a waste water stream comprising a major amount of water in the recovered liquid acetone stream, and; iii) a waste organic stream comprising a major portion of: the impurity, and the ketonization byproducts present in the recovered liquid acetone stream.

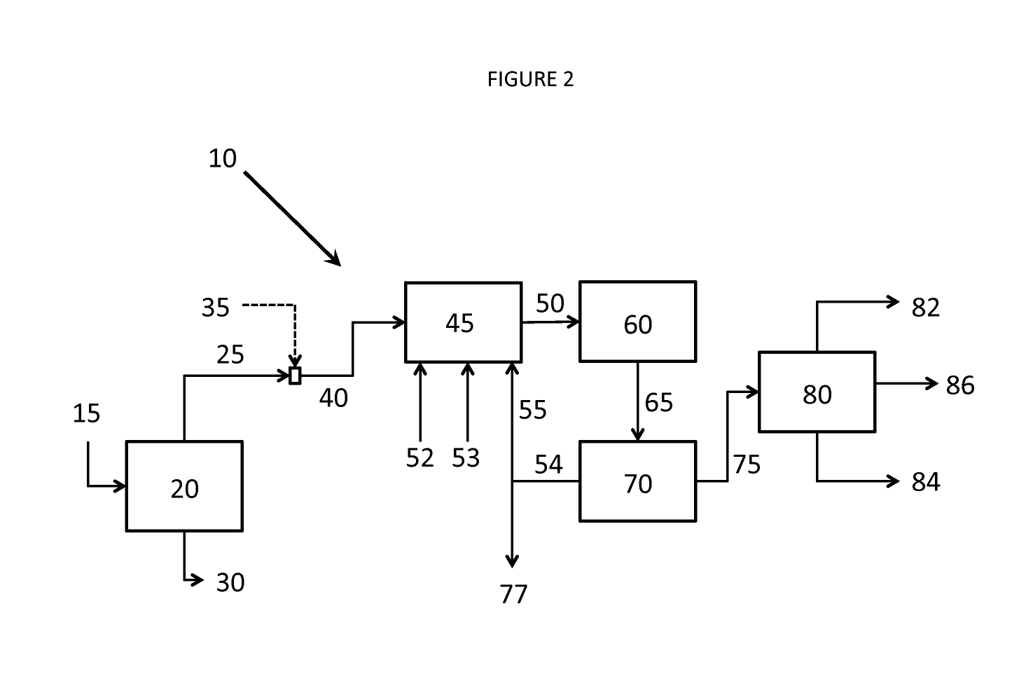

With reference to FIG. 2, the various steps of a method for ketonization and purification are described in greater detail. The ketonization process 10 includes feeding an acetyl stream 15 comprising acetic acid, an impurity comprising at least one acetic acid azeotrope-forming compound and 0-50 weight % water, based on the weight of the feed stream, to a vaporization unit 20, wherein a fraction, typically 75 to 99%, of the acetyl feed stream 15 is vaporized by boiling against steam, to produce vaporized acetyl stream 25. A portion of the feed acid that is not vaporized is removed from the vaporization unit 20 as sludge via line 30. Vaporized acetyl stream 25 is optionally mixed with steam 35 for further dilution of the feed acid to produce vaporized wet acid feed mixture 40. Vaporized wet acid feed mixture 40 is further superheated to the desired reaction inlet temperature in a feed superheater furnace 45 to produce superheated feed mixture 50. Heat is provided to the furnace by combustion of fuel 52 with an oxygen-containing stream 53, which may be diluted for temperature control by at least a portion of by-product carbon dioxide stream 54 via conduit 55. The superheated acid mixture 50 is passed through ketonization reactor 60, wherein the acetic acid and other reactive feed molecules, if present, are converted over a heterogeneous ketonization catalyst to a crude product mixture 65 comprising acetone, water, carbon dioxide, unreacted acetic acid, the impurity having at least one acetic acid azeotrope-forming compound, and other minor by-products. Crude product mixture 65, is cooled and separated in recovery zone 70 to produce a crude liquid acetone stream 75, comprising the majority of the acetone, water, impurities and heavy by-products; and gaseous off-gas stream 54 comprising carbon dioxide, isobutylene, methane, hydrogen, other minor VOC's, and traces of acetone and higher by-products. Gaseous off-gas stream 54, may be sent in its entirety via conduit 55 to the superheater or furnace 45, or a portion emitted directly via conduit 77. Typically during, normal operation all of stream 54 will be sent to the superheater 45 for combustion of volatile organic compounds (VOCs), although at start up, or during furnace upsets, a fraction or all of stream 54 may exit the process via stream 77 without further treatment. The crude liquid acetone stream 75 is further purified in distillation zone 80 to produce a purified acetone stream 82 comprising at least 95 weight % acetone and a minor amount of: water, an impurity having at least one azeotrope-forming compound, and ketonization byproducts present in the crude product mixture; a waste water stream 84, comprising water from the acid feed 15, any added steam 35, water created in the ketonization reactor, as well as any water added in the recovery zone 70; and a waste organic stream 86, comprising a major portion of the impurity having at least one acetic acid azeotrope-forming compound, and the ketonization byproducts present in the recovered liquid acetone stream.

Advantageously, the acetic acid comprising the acetyl feed stream 15 can be, but is not limited to, a byproduct from one or more of the processes discussed above, i.e., acetylation of a compound selected from an alcohol, a polyol, cellulose, an amine, carboxylic acid, and an aromatic compound by contacting the compound with acetic anhydride. For example, the acetic acid utilized can be a byproduct from one or more of the following processes: acetylation of wood; acetylation of alcohols with acetic anhydride to form esters; carbonylation of methanol and methyl acetate to form acetic acid and acetic anhydride; preparation of 2,2,4,4-tetramethyl-1,3-cyclobutanediol from isobutyric anhydride via acetic anhydride exchange; preparation of ketenes and diketene from acetic acid; polymerization reactions, such as condensation of phenyl acetate monomers to produce polyesters or polycarbonates; preparation of fine chemicals and pharmaceuticals; preparation of carboxylic acid anhydrides from their corresponding acids by exchange with acetic anhydride; and acylation reactions. Examples for fine chemical and pharmaceutical products include but are not limited, to industrial production of ibuprofen and liquid crystal polymers.

It will be recognized that acetic acid from other sources may also be equally suitable for use in the process of the present invention. Such sources include, but are not limited to, acetaldehyde oxidation, ethylene oxidation, oxidative fermentation, and anaerobic biomass fermentation, producer waters from oil and gas production, Fischer-Tropsch derived acetic acid, and carbonaceous reforming.

The byproduct acetyl feed stream 15, containing a mixture of acetic acid, an impurity comprising at least one acetic acid azeotrope or pinch-forming impurity, and optionally, acetic anhydride, can be mixed with water to hydrolyze any acetic anhydride that is present to produce a wet acetic acid feed stream. The acetyl feed stream typically contain from about 40 to about 99 weight % acetic acid, up to about 50 weight % impurities, and optionally up to about 10 weight % acetic anhydride, wherein the weight percentages are based on total constituents in the feed stream. Accordingly, the acetyl feed stream 15 can have from about 40 to about 99 weight % acetic acid, or from about 70 to about 99 weight % acetic acid, or from about 86 to about 99 weight % acetic acid, or about 87 to about 99 weight % acetic acid, or about 88 to about 99 weight % acetic acid, or about 89 to about 99 weight % acetic acid, or about 90 to about 99 weight % acetic acid, or about 91 to about 99 weight % acetic acid, or about 92 to about 99 weight % acetic acid, or about 93 to about 99 weight % acetic acid, or about 94 to about 99 weight % acetic acid, or about 95 to about 99 weight % acetic acid, or about 96 to about 99 weight % acetic acid, or about 97 to about 99 weight % acetic acid, or about 98 to about 99 weight % acetic acid; and up to about 50 weight % impurities, or from about 100 ppm to about 50 weight % impurities, or from about 200 ppm to about 50 weight % impurities, or from about 500 ppm to about 50 weight % impurities, or from about 1000 ppm to about 50 weight % impurities, or from about 5000 ppm to about 50 weight % impurities, or from about 1 weight % to about 50 weight % impurities, or from about 2 weight % to about 50 weight % impurities, or from about 3 weight % to about 50 weight % impurities, or from about 4 weight % to about 50 weight % impurities, or from about 5 weight % to about 50 weight % impurities, or from about 6 weight % to about 50 weight % impurities, or from about 7 weight % to about 50 weight % impurities, or from about 8 weight % to about 50 weight % impurities, or from about 9 weight % to about 50 weight % impurities, or from about 10 weight % to about 50 weight % impurities, or from about 11 weight % to about 50 weight % impurities, or from about 12 weight % to about 50 weight % impurities, or from about 13 weight % to about 50 weight % impurities, or from about 14 weight % to about 50 weight % impurities; or from about 15 weight % to about 50 weight % impurities, or from about 16 weight % to about 50 weight % impurities, or from about 17 weight % to about 50 weight % impurities, or from about 18 weight % to about 50 weight % impurities, or from about 19 weight % to about 50 weight % impurities, or from about 20 weight % to about 50 weight % impurities, or from about 21 weight % to about 50 weight % impurities, or from about 22 weight % to about 50 weight % impurities, or from about 23 weight % to about 50 weight % impurities, or from about 24 weight % to about 50 weight % impurities, or from about 25 weight % to about 50 weight % impurities; 26 weight % to about 50 weight % impurities, or from about 27 weight % to about 50 weight % impurities, or from about 28 weight % to about 50 weight % impurities, or from about 29 weight % to about 50 weight % impurities, or from about 30 weight % to about 50 weight % impurities, or from about 31 weight % to about 50 weight % impurities, or from about 32 weight % to about 50 weight % impurities, or from about 33 weight % to about 50 weight % impurities, or from about 34 weight % to about 50 weight % impurities, or from about 35 weight % to about 50 weight % impurities, or from about 36 weight % to about 50 weight % impurities, or from about 37 weight % to about 50 weight % impurities, or from about 38 weight % to about 50 weight % impurities; or from about 39 weight % to about 50 weight % impurities, or from about 40 weight % to about 50 weight % impurities, or from about 41 weight % to about 50 weight % impurities, or from about 42 weight % to about 43 weight % impurities, or from about 44 weight % to about 50 weight % impurities, or from about 45 weight % to about 50 weight % impurities, or from about 46 weight % to about 50 weight % impurities, or from about 47 weight % to about 50 weight % impurities, or from about 48 weight % to about 50 weight % impurities, or from about 49 weight % to about 50 weight % impurities.

Optionally, the byproduct acetyl feed stream 15, may have from 200 ppm to about 15 weight % impurities, or from about 500 ppm to about 15 weight % impurities, or from about 1000 ppm to about 15 weight % impurities, or from about 5000 ppm to about 15 weight percent, or from 1 to about 15 weight % impurities, or from 2 to about 15 weight % impurities, or from 3 to about 15 weight % impurities, or from 4 to about 15 weight % impurities, or from 5 to about 15 weight % impurities, or from 6 to about 15 weight % impurities, or from 7 to about 15 weight % impurities, or from 8 to about 15 weight % impurities, or from 9 to about 15 weight % impurities, or from 10 to about 15 weight % impurities, or from 11 to about 15 weight % impurities, or from 12 to about 15 weight % impurities, or from 13 to about 15 weight % impurities, or from 14 to about 15 weight % impurities, or from 200 ppm to about 14 weight % impurities, or from about 500 ppm to about 14 weight % impurities, or from about 1000 ppm to about 14 weight % impurities, or from about 5000 ppm to about 14 weight percent, or from 1 to about 14 weight % impurities, or from 2 to about 14 weight % impurities, or from 3 to about 14 weight % impurities, or from 4 to about 14 weight % impurities, or from 5 to about 14 weight % impurities, or from 6 to about 14 weight % impurities, or from 7 to about 14 weight % impurities, or from 8 to about 14 weight % impurities, or from 9 to about 14 weight % impurities, or from 10 to about 14 weight % impurities, or from 11 to about 14 weight % impurities, or from 12 to about 14 weight % impurities, or from 13 to about 14 weight % impurities, or from 200 ppm to about 13 weight % impurities, or from about 500 ppm to about 15 weight % impurities, or from about 1000 ppm to about 13 weight % impurities, or from about 5000 ppm to about 13 weight percent, or from 1 to about 13 weight % impurities, or from 2 to about 13 weight % impurities, or from 3 to about 13 weight % impurities, or from 4 to about 13 weight % impurities, or from 5 to about 13 weight % impurities, or from 6 to about 13 weight % impurities, or from 7 to about 13 weight % impurities, or from 8 to about 13 weight % impurities, or from 9 to about 13 weight % impurities, or from 10 to about 13 weight % impurities, or from 11 to about 13 weight % impurities, or from 12 to about 13 weight % impurities, or from 200 ppm to about 12 weight % impurities, or from about 500 ppm to about 12 weight % impurities, or from about 1000 ppm to about 12 weight % impurities, or from about 5000 ppm to about 12 weight percent, or from 1 to about 12 weight % impurities, or from 2 to about 12 weight % impurities, or from 3 to about 12 weight % impurities, or from 4 to about 12 weight % impurities, or from 5 to about 12 weight % impurities, or from 6 to about 12 weight % impurities, or from 7 to about 12 weight % impurities, or from 8 to about 12 weight % impurities, or from 9 to about 12 weight % impurities, or from 10 to about 12 weight % impurities, or from 11 to about 12 weight % impurities, or from 200 ppm to about 11 weight % impurities, or from about 500 ppm to about 11 weight % impurities, or from about 1000 ppm to about 11 weight % impurities, or from about 5000 ppm to about 11 weight percent, or from 1 to about 11 weight % impurities, or from 2 to about 11 weight % impurities, or from 3 to about 11 weight % impurities, or from 4 to about 11 weight % impurities, or from 5 to about 11 weight % impurities, or from 6 to about 11 weight % impurities, or from 7 to about 11 weight % impurities, or from 8 to about 11 weight % impurities, or from 9 to about 11 weight % impurities, or from 10 to about 11 weight % impurities, or from 200 ppm to about 10 weight % impurities, or from about 500 ppm to about 10 weight % impurities, or from about 1000 ppm to about 10 weight % impurities, or from about 5000 ppm to about 10 weight percent, or from 1 to about 10 weight % impurities, or from 2 to about 10 weight % impurities, or from 3 to about 10 weight % impurities, or from 4 to about 10 weight % impurities, or from 5 to about 10 weight % impurities, or from 6 to about 10 weight % impurities, or from 7 to about 10 weight % impurities, or from 8 to about 10 weight % impurities, or from 9 to about 10 weight % impurities, or from 200 ppm to about 9 weight % impurities, or from about 500 ppm to about 9 weight % impurities, or from about 1000 ppm to about 9 weight % impurities, or from about 5000 ppm to about 9 weight percent, or from 1 to about 9 weight % impurities, or from 2 to about 9 weight % impurities, or from 3 to about 9 weight % impurities, or from 4 to about 9 weight % impurities, or from 5 to about 9 weight % impurities, or from 6 to about 9 weight % impurities, or from 7 to about 9 weight % impurities, or from 8 to about 9 weight % impurities, or from 200 ppm to about 8 weight % impurities, or from about 500 ppm to about 8 weight % impurities, or from about 1000 ppm to about 8 weight % impurities, or from about 5000 ppm to about 8 weight percent, or from 1 to about 8 weight % impurities, or from 2 to about 8 weight % impurities, or from 3 to about 8 weight % impurities, or from 4 to about 8 weight % impurities, or from 5 to about 8 weight % impurities, or from 6 to about 8 weight % impurities, or from 7 to about 8 weight % impurities, or from 200 ppm to about 7 weight % impurities, or from about 500 ppm to about 7 weight % impurities, or from about 1000 ppm to about 7 weight % impurities, or from about 5000 ppm to about 7 weight percent, or from 1 to about 7 weight % impurities, or from 2 to about 7 weight % impurities, or from 3 to about 7 weight % impurities, or from 4 to about 7 weight % impurities, or from 5 to about 7 weight % impurities, or from 6 to about 7 weight % impurities, or from 200 ppm to about 6 weight % impurities, or from about 500 ppm to about 6 weight % impurities, or from about 1000 ppm to about 6 weight % impurities, or from about 5000 ppm to about 6 weight percent, or from 1 to about 6 weight % impurities, or from 2 to about 6 weight % impurities, or from 3 to about 6 weight % impurities, or from 4 to about 6 weight % impurities, or from 200 ppm to about 5 weight % impurities, or from about 500 ppm to about 5 weight % impurities, or from about 1000 ppm to about 5 weight % impurities, or from about 5000 ppm to about 5 weight percent, or from 1 to about 5 weight % impurities, or from 2 to about 5 weight % impurities, or from 3 to about 5 weight % impurities, or from 4 to about 5 weight % impurities, or from 200 ppm to about 4 weight % impurities, or from about 500 ppm to about 4 weight % impurities, or from about 1000 ppm to about 4 weight % impurities, or from about 5000 ppm to about 4 weight percent, or from 1 to about 4 weight % impurities, or from 2 to about 4 weight % impurities, or from 3 to about 4 weight % impurities, or from 200 ppm to about 3 weight % impurities, or from about 500 ppm to about 3 weight % impurities, or from about 1000 ppm to about 3 weight % impurities, or from about 5000 ppm to about 3 weight percent, or from 1 to about 3 weight % impurities, or from 2 to about 3 weight % impurities, or from 200 ppm to about 2 weight % impurities, or from about 500 ppm to about 2 weight % impurities, or from about 1000 ppm to about 2 weight % impurities, or from about 5000 ppm to about 2 weight percent, or from 1 to about 2 weight % impurities, wherein the weight percentages presented above are based on the total weight of the constituents of the feed stream 15.

Typically, due to excessive fouling of the vaporizer 20, superheater 45, and reactor 60, it is not desirable to feed anhydrous acetic acid or acetic anhydride. Rather, the acetyl feed stream 15 is mixed with sufficient water to hydrolyze any acetic anhydride that may be present prior to introducing the acetyl feed stream 15 to the vaporizer 20. Accordingly, the feed stream 15 can be mixed with water to bring the final concentration of water in the acetyl feed stream 15 up to about 80 weight % water, or from about 10 weight % to about 80 weight % water, or from about 15 weight % to about 80 weight % water, or from about 20 weight % to about 80 weight % water, or from about 25 weight % to about 80 weight % water, or from about 30 weight % to about 80 weight % water, or from about 35 weight % to about 80 weight % water, or from about 40 weight % to about 80 weight % water, or from about 45 weight % to about 80 weight % water, or from about 50 weight % to about 80 weight % water, or from about 55 weight % to about 80 weight % water, or from about 60 weight % to about 80 weight % water, or from about 65 weight % to about 80 weight % water, or from about 70 weight % to about 80 weight % water, or from about 75 weight % to about 80 weight % water, wherein the weight percentage is based on the total weight of the feed stream 15.

The feed stream 15 may optionally further include up to about 75 weight % water, or up to about 70 weight % water, or up to about 65 weight % water, or up to about 60 weight % water, or up to about 55 weight % water, or up to about 50 weight % water, or up to about 45 weight % water, or up to about 40 weight % water, or up to about 35 weight % water, or up to about 30 weight % water, or up to about 25 weight % water, or up to about 20 weight % water, or up to about 15 weight % water, or up to about 10 weight % water, wherein the weight percentage is based on the total weight of the constituents of the feed stream 15. It should be generally understood that in the ranges specified above the term “up to” includes from 0 to the delineated end point, and includes all ranges in between. Such ranges include 0 to 80, 1 to 80, 2 to 80, 3 to 80, 4 to 80, 5 to 80, 6 to 80, 7 to 80, 8 to 80, 9 to 80, 10 to 80, 11 to 80, 12 to 80, 13 to 80, 14 to 80, 15 to 80, 16 to 80, 17 to 80, 18 to 80, 19 to 80, 20 to 80, 21 to 80, 22 to 80, 23 to 80, 24 to 80, 25 to 80, 26 to 80, 27 to 80, 28 to 80, 29 to 80, 30 to 80, 31 to 80, 32 to 80, 33 to 80, 34 to 80, 35 to 80, 36 to 80, 37 to 80, 38 to 80, 39 to 80, 40 to 80, 41 to 80, 42 to 80, 43 to 80, 44 to 80, 45 to 80, 46 to 80, 47 to 80, 48 to 80, 49 to 80, 50 to 80, 51 to 80, 52 to 80, 53 to 80, 54 to 80, 55 to 80, 56 to 80, 57 to 80, 58 to 80, 59 to 80, 60 to 80, 61 to 80, 62 to 80, 63 to 80, 64 to 80, 65 to 80, 66 to 80, 67 to 80, 68 to 80, 69 to 80, 70 to 80, 71 to 80, 72 to 80, 73 to 80, 74 to 80, 75 to 80, 76 to 80, 77 to 80, 78 to 80, 79 to 80, 0 to 79, 1 to 79, 2 to 79, 3 to 79, 4 to 79, 5 to 79, 6 to 79, 7 to 79, 8 to 79, 9 to 79, 10 to 79, 11 to 79, 12 to 79, 13 to 79, 14 to 79, 15 to 79, 16 to 79, 17 to 79, 18 to 79, 19 to 79, 20 to 79, 21 to 79, 22 to 79, 23 to 79, 24 to 79, 25 to 79, 26 to 79, 27 to 79, 28 to 79, 29 to 79, 30 to 79, 31 to 79, 32 to 79, 33 to 79, 34 to 79, 35 to 79, 36 to 79, 37 to 79, 38 to 79, 39 to 79, 40 to 79, 41 to 79, 42 to 79, 43 to 79, 44 to 79, 45 to 79, 46 to 79, 47 to 79, 48 to 79, 49 to 79, 50 to 79, 51 to 79, 52 to 79, 53 to 79, 54 to 79, 55 to 79, 56 to 79, 57 to 79, 58 to 79, 59 to 79, 60 to 79, 61 to 79, 62 to 79, 63 to 79, 64 to 79, 65 to 79, 66 to 79, 67 to 79, 68 to 79, 69 to 79, 70 to 79, 71 to 79, 72 to 79, 73 to 79, 74 to 79, 75 to 79, 76 to 79, 77 to 79, 78 to 79, 0 to 78, 1 to 78, 2 to 78, 3 to 78, 4 to 78, 5 to 78, 6 to 78, 7 to 78, 8 to 78, 9 to 78, 10 to 78, 11 to 78, 12 to 78, 13 to 78, 14 to 78, 15 to 78, 16 to 78, 17 to 78, 18 to 78, 19 to 78, 20 to 78, 21 to 78, 22 to 78, 23 to 78, 24 to 78, 25 to 78, 26 to 78, 27 to 78, 28 to 78, 29 to 78, 30 to 78, 31 to 78, 32 to 78, 33 to 78, 34 to 78, 35 to 78, 36 to 78, 37 to 78, 38 to 78, 39 to 78, 40 to 78, 41 to 78, 42 to 78, 43 to 78, 44 to 78, 45 to 78, 46 to 78, 47 to 78, 48 to 78, 49 to 78, 50 to 78, 51 to 78, 52 to 78, 53 to 78, 54 to 78, 55 to 78, 56 to 78, 57 to 78, 58 to 78, 59 to 78, 60 to 78, 61 to 78, 62 to 78, 63 to 78, 64 to 78, 65 to 78, 66 to 78, 67 to 78, 68 to 78, 69 to 78, 70 to 78, 71 to 78, 72 to 78, 73 to 78, 74 to 78, 75 to 78, 76 to 78, 77 to 78, 0 to 77, 1 to 77, 2 to 77, 3 to 77, 4 to 77, 5 to 77, 6 to 77, 7 to 77, 8 to 77, 9 to 77, 10 to 77, 11 to 77, 12 to 77, 13 to 77, 14 to 77, 15 to 77, 16 to 77, 17 to 77, 18 to 77, 19 to 77, 20 to 77, 21 to 77, 22 to 77, 23 to 77, 24 to 77, 25 to 77, 26 to 77, 27 to 77, 28 to 77, 29 to 77, 30 to 77, 31 to 77, 32 to 77, 33 to 77, 34 to 77, 35 to 77, 36 to 77, 37 to 77, 38 to 77, 39 to 77, 40 to 77, 41 to 77, 42 to 77, 43 to 77, 44 to 77, 45 to 77, 46 to 77, 47 to 77, 48 to 77, 49 to 77, 50 to 77, 51 to 77, 52 to 77, 53 to 77, 54 to 77, 55 to 77, 56 to 77, 57 to 77, 58 to 77, 59 to 77, 60 to 77, 61 to 77, 62 to 77, 63 to 77, 64 to 77, 65 to 77, 66 to 77, 67 to 77, 68 to 77, 69 to 77, 70 to 77, 71 to 77, 72 to 77, 73 to 77, 74 to 77, 75 to 77, 76 to 77, 0 to 76, 1 to 76, 2 to 76, 3 to 76, 4 to 76, 5 to 76, 6 to 76, 7 to 76, 8 to 76, 9 to 76, 10 to 76, 11 to 76, 12 to 76, 13 to 76, 14 to 76, 15 to 76, 16 to 76, 17 to 76, 18 to 76, 19 to 76, 20 to 76, 21 to 76, 22 to 76, 23 to 76, 24 to 76, 25 to 76, 26 to 76, 27 to 76, 28 to 76, 29 to 76, 30 to 76, 31 to 76, 32 to 76, 33 to 76, 34 to 76, 35 to 76, 36 to 76, 37 to 76, 38 to 76, 39 to 76, 40 to 76, 41 to 76, 42 to 76, 43 to 76, 44 to 76, 45 to 76, 46 to 76, 47 to 76, 48 to 76, 49 to 76, 50 to 76, 51 to 76, 52 to 76, 53 to 76, 54 to 76, 55 to 76, 56 to 76, 57 to 76, 58 to 76, 59 to 76, 60 to 76, 61 to 76, 62 to 76, 63 to 76, 64 to 76, 65 to 76, 66 to 76, 67 to 76, 68 to 76, 69 to 76, 70 to 76, 71 to 76, 72 to 76, 73 to 76, 74 to 76, 75 to 76, 0 to 75, 1 to 75, 2 to 75, 3 to 75, 4 to 75, 5 to 75, 6 to 75, 7 to 75, 8 to 75, 9 to 75, 10 to 75, 11 to 75, 12 to 75, 13 to 75, 14 to 75, 15 to 75, 16 to 75, 17 to 75, 18 to 75, 19 to 75, 20 to 75, 21 to 75, 22 to 75, 23 to 75, 24 to 75, 25 to 75, 26 to 75, 27 to 75, 28 to 75, 29 to 75, 30 to 75, 31 to 75, 32 to 75, 33 to 75, 34 to 75, 35 to 75, 36 to 75, 37 to 75, 38 to 75, 39 to 75, 40 to 75, 41 to 75, 42 to 75, 43 to 75, 44 to 75, 45 to 75, 46 to 75, 47 to 75, 48 to 75, 49 to 75, 50 to 75, 51 to 75, 52 to 75, 53 to 75, 54 to 75, 55 to 75, 56 to 75, 57 to 75, 58 to 75, 59 to 75, 60 to 75, 61 to 75, 62 to 75, 63 to 75, 64 to 75, 65 to 75, 66 to 75, 67 to 75, 68 to 75, 69 to 75, 70 to 75, 71 to 75, 72 to 75, 73 to 75, 74 to 75, 0 to 74, 1 to 74, 2 to 74, 3 to 74, 4 to 74, 5 to 74, 6 to 74, 7 to 74, 8 to 74, 9 to 74, 10 to 74, 11 to 74, 12 to 74, 13 to 74, 14 to 74, 15 to 74, 16 to 74, 17 to 74, 18 to 74, 19 to 74, 20 to 74, 21 to 74, 22 to 74, 23 to 74, 24 to 74, 25 to 74, 26 to 74, 27 to 74, 28 to 74, 29 to 74, 30 to 74, 31 to 74, 32 to 74, 33 to 74, 34 to 74, 35 to 74, 36 to 74, 37 to 74, 38 to 74, 39 to 74, 40 to 74, 41 to 74, 42 to 74, 43 to 74, 44 to 74, 45 to 74, 46 to 74, 47 to 74, 48 to 74, 49 to 74, 50 to 74, 51 to 74, 52 to 74, 53 to 74, 54 to 74, 55 to 74, 56 to 74, 57 to 74, 58 to 74, 59 to 74, 60 to 74, 61 to 74, 62 to 74, 63 to 74, 64 to 74, 65 to 74, 66 to 74, 67 to 74, 68 to 74, 69 to 74, 70 to 74, 71 to 74, 72 to 74, 73 to 74, 0 to 73, 1 to 73, 2 to 73, 3 to 73, 4 to 73, 5 to 73, 6 to 73, 7 to 73, 8 to 73, 9 to 73, 10 to 73, 11 to 73, 12 to 73, 13 to 73, 14 to 73, 15 to 73, 16 to 73, 17 to 73, 18 to 73, 19 to 73, 20 to 73, 21 to 73, 22 to 73, 23 to 73, 24 to 73, 25 to 73, 26 to 73, 27 to 73, 28 to 73, 29 to 73, 30 to 73, 31 to 73, 32 to 73, 33 to 73, 34 to 73, 35 to 73, 36 to 73, 37 to 73, 38 to 73, 39 to 73, 40 to 73, 41 to 73, 42 to 73, 43 to 73, 44 to 73, 45 to 73, 46 to 73, 47 to 73, 48 to 73, 49 to 73, 50 to 73, 51 to 73, 52 to 73, 53 to 73, 54 to 73, 55 to 73, 56 to 73, 57 to 73, 58 to 73, 59 to 73, 60 to 73, 61 to 73, 62 to 73, 63 to 73, 64 to 73, 65 to 73, 66 to 73, 67 to 73, 68 to 73, 69 to 73, 70 to 73, 71 to 73, 72 to 73, 0 to 72, 1 to 72, 2 to 72, 3 to 72, 4 to 72, 5 to 72, 6 to 72, 7 to 72, 8 to 72, 9 to 72, 10 to 72, 11 to 72, 12 to 72, 13 to 72, 14 to 72, 15 to 72, 16 to 72, 17 to 72, 18 to 72, 19 to 72, 20 to 72, 21 to 72, 22 to 72, 23 to 72, 24 to 72, 25 to 72, 26 to 72, 27 to 72, 28 to 72, 29 to 72, 30 to 72, 31 to 72, 32 to 72, 33 to 72, 34 to 72, 35 to 72, 36 to 72, 37 to 72, 38 to 72, 39 to 72, 40 to 72, 41 to 72, 42 to 72, 43 to 72, 44 to 72, 45 to 72, 46 to 72, 47 to 72, 48 to 72, 49 to 72, 50 to 72, 51 to 72, 52 to 72, 53 to 72, 54 to 72, 55 to 72, 56 to 72, 57 to 72, 58 to 72, 59 to 72, 60 to 72, 61 to 72, 62 to 72, 63 to 72, 64 to 72, 65 to 72, 66 to 72, 67 to 72, 68 to 72, 69 to 72, 70 to 72, 71 to 72, 0 to 71, 1 to 71, 2 to 71, 3 to 71, 4 to 71, 5 to 71, 6 to 71, 7 to 71, 8 to 71, 9 to 71, 10 to 71, 11 to 71, 12 to 71, 13 to 71, 14 to 71, 15 to 71, 16 to 71, 17 to 71, 18 to 71, 19 to 71, 20 to 71, 21 to 71, 22 to 71, 23 to 71, 24 to 71, 25 to 71, 26 to 71, 27 to 71, 28 to 71, 29 to 71, 30 to 71, 31 to 71, 32 to 71, 33 to 71, 34 to 71, 35 to 71, 36 to 71, 37 to 71, 38 to 71, 39 to 71, 40 to 71, 41 to 71, 42 to 71, 43 to 71, 44 to 71, 45 to 71, 46 to 71, 47 to 71, 48 to 71, 49 to 71, 50 to 71, 51 to 71, 52 to 71, 53 to 71, 54 to 71, 55 to 71, 56 to 71, 57 to 71, 58 to 71, 59 to 71, 60 to 71, 61 to 71, 62 to 71, 63 to 71, 64 to 71, 65 to 71, 66 to 71, 67 to 71, 68 to 71, 69 to 71, 70 to 71, 0 to 70, 1 to 70, 2 to 70, 3 to 70, 4 to 70, 5 to 70, 6 to 70, 7 to 70, 8 to 70, 9 to 70, 10 to 70, 11 to 70, 12 to 70, 13 to 70, 14 to 70, 15 to 70, 16 to 70, 17 to 70, 18 to 70, 19 to 70, 20 to 70, 21 to 70, 22 to 70, 23 to 70, 24 to 70, 25 to 70, 26 to 70, 27 to 70, 28 to 70, 29 to 70, 30 to 70, 31 to 70, 32 to 70, 33 to 70, 34 to 70, 35 to 70, 36 to 70, 37 to 70, 38 to 70, 39 to 70, 40 to 70, 41 to 70, 42 to 70, 43 to 70, 44 to 70, 45 to 70, 46 to 70, 47 to 70, 48 to 70, 49 to 70, 50 to 70, 51 to 70, 52 to 70, 53 to 70, 54 to 70, 55 to 70, 56 to 70, 57 to 70, 58 to 70, 59 to 70, 60 to 70, 61 to 70, 62 to 70, 63 to 70, 64 to 70, 65 to 70, 66 to 70, 67 to 70, 68 to 70, 69 to 70, 0 to 69, 1 to 69, 2 to 69, 3 to 69, 4 to 69, 5 to 69, 6 to 69, 7 to 69, 8 to 69, 9 to 69, 10 to 69, 11 to 69, 12 to 69, 13 to 69, 14 to 69, 15 to 69, 16 to 69, 17 to 69, 18 to 69, 19 to 69, 20 to 69, 21 to 69, 22 to 69, 23 to 69, 24 to 69, 25 to 69, 26 to 69, 27 to 69, 28 to 69, 29 to 69, 30 to 69, 31 to 69, 32 to 69, 33 to 69, 34 to 69, 35 to 69, 36 to 69, 37 to 69, 38 to 69, 39 to 69, 40 to 69, 41 to 69, 42 to 69, 43 to 69, 44 to 69, 45 to 69, 46 to 69, 47 to 69, 48 to 69, 49 to 69, 50 to 69, 51 to 69, 52 to 69, 53 to 69, 54 to 69, 55 to 69, 56 to 69, 57 to 69, 58 to 69, 59 to 69, 60 to 69, 61 to 69, 62 to 69, 63 to 69, 64 to 69, 65 to 69, 66 to 69, 67 to 69, 68 to 69, 0 to 68, 1 to 68, 2 to 68, 3 to 68, 4 to 68, 5 to 68, 6 to 68, 7 to 68, 8 to 68, 9 to 68, 10 to 68, 11 to 68, 12 to 68, 13 to 68, 14 to 68, 15 to 68, 16 to 68, 17 to 68, 18 to 68, 19 to 68, 20 to 68, 21 to 68, 22 to 68, 23 to 68, 24 to 68, 25 to 68, 26 to 68, 27 to 68, 28 to 68, 29 to 68, 30 to 68, 31 to 68, 32 to 68, 33 to 68, 34 to 68, 35 to 68, 36 to 68, 37 to 68, 38 to 68, 39 to 68, 40 to 68, 41 to 68, 42 to 68, 43 to 68, 44 to 68, 45 to 68, 46 to 68, 47 to 68, 48 to 68, 49 to 68, 50 to 68, 51 to 68, 52 to 68, 53 to 68, 54 to 68, 55 to 68, 56 to 68, 57 to 68, 58 to 68, 59 to 68, 60 to 68, 61 to 68, 62 to 68, 63 to 68, 64 to 68, 65 to 68, 66 to 68, 67 to 68, 0 to 67, 1 to 67, 2 to 67, 3 to 67, 4 to 67, 5 to 67, 6 to 67, 7 to 67, 8 to 67, 9 to 67, 10 to 67, 11 to 67, 12 to 67, 13 to 67, 14 to 67, 15 to 67, 16 to 67, 17 to 67, 18 to 67, 19 to 67, 20 to 67, 21 to 67, 22 to 67, 23 to 67, 24 to 67, 25 to 67, 26 to 67, 27 to 67, 28 to 67, 29 to 67, 30 to 67, 31 to 67, 32 to 67, 33 to 67, 34 to 67, 35 to 67, 36 to 67, 37 to 67, 38 to 67, 39 to 67, 40 to 67, 41 to 67, 42 to 67, 43 to 67, 44 to 67, 45 to 67, 46 to 67, 47 to 67, 48 to 67, 49 to 67, 50 to 67, 51 to 67, 52 to 67, 53 to 67, 54 to 67, 55 to 67, 56 to 67, 57 to 67, 58 to 67, 59 to 67, 60 to 67, 61 to 67, 62 to 67, 63 to 67, 64 to 67, 65 to 67, 66 to 67, 0 to 66, 1 to 66, 2 to 66, 3 to 66, 4 to 66, 5 to 66, 6 to 66, 7 to 66, 8 to 66, 9 to 66, 10 to 66, 11 to 66, 12 to 66, 13 to 66, 14 to 66, 15 to 66, 16 to 66, 17 to 66, 18 to 66, 19 to 66, 20 to 66, 21 to 66, 22 to 66, 23 to 66, 24 to 66, 25 to 66, 26 to 66, 27 to 66, 28 to 66, 29 to 66, 30 to 66, 31 to 66, 32 to 66, 33 to 66, 34 to 66, 35 to 66, 36 to 66, 37 to 66, 38 to 66, 39 to 66, 40 to 66, 41 to 66, 42 to 66, 43 to 66, 44 to 66, 45 to 66, 46 to 66, 47 to 66, 48 to 66, 49 to 66, 50 to 66, 51 to 66, 52 to 66, 53 to 66, 54 to 66, 55 to 66, 56 to 66, 57 to 66, 58 to 66, 59 to 66, 60 to 66, 61 to 66, 62 to 66, 63 to 66, 64 to 66, 65 to 66, 0 to 65, 1 to 65, 2 to 65, 3 to 65, 4 to 65, 5 to 65, 6 to 65, 7 to 65, 8 to 65, 9 to 65, 10 to 65, 11 to 65, 12 to 65, 13 to 65, 14 to 65, 15 to 65, 16 to 65, 17 to 65, 18 to 65, 19 to 65, 20 to 65, 21 to 65, 22 to 65, 23 to 65, 24 to 65, 25 to 65, 26 to 65, 27 to 65, 28 to 65, 29 to 65, 30 to 65, 31 to 65, 32 to 65, 33 to 65, 34 to 65, 35 to 65, 36 to 65, 37 to 65, 38 to 65, 39 to 65, 40 to 65, 41 to 65, 42 to 65, 43 to 65, 44 to 65, 45 to 65, 46 to 65, 47 to 65, 48 to 65, 49 to 65, 50 to 65, 51 to 65, 52 to 65, 53 to 65, 54 to 65, 55 to 65, 56 to 65, 57 to 65, 58 to 65, 59 to 65, 60 to 65, 61 to 65, 62 to 65, 63 to 65, 64 to 65, 0 to 64, 1 to 64, 2 to 64, 3 to 64, 4 to 64, 5 to 64, 6 to 64, 7 to 64, 8 to 64, 9 to 64, 10 to 64, 11 to 64, 12 to 64, 13 to 64, 14 to 64, 15 to 64, 16 to 64, 17 to 64, 18 to 64, 19 to 64, 20 to 64, 21 to 64, 22 to 64, 23 to 64, 24 to 64, 25 to 64, 26 to 64, 27 to 64, 28 to 64, 29 to 64, 30 to 64, 31 to 64, 32 to 64, 33 to 64, 34 to 64, 35 to 64, 36 to 64, 37 to 64, 38 to 64, 39 to 64, 40 to 64, 41 to 64, 42 to 64, 43 to 64, 44 to 64, 45 to 64, 46 to 64, 47 to 64, 48 to 64, 49 to 64, 50 to 64, 51 to 64, 52 to 64, 53 to 64, 54 to 64, 55 to 64, 56 to 64, 57 to 64, 58 to 64, 59 to 64, 60 to 64, 61 to 64, 62 to 64, 63 to 64, 0 to 63, 1 to 63, 2 to 63, 3 to 63, 4 to 63, 5 to 63, 6 to 63, 7 to 63, 8 to 63, 9 to 63, 10 to 63, 11 to 63, 12 to 63, 13 to 63, 14 to 63, 15 to 63, 16 to 63, 17 to 63, 18 to 63, 19 to 63, 20 to 63, 21 to 63, 22 to 63, 23 to 63, 24 to 63, 25 to 63, 26 to 63, 27 to 63, 28 to 63, 29 to 63, 30 to 63, 31 to 63, 32 to 63, 33 to 63, 34 to 63, 35 to 63, 36 to 63, 37 to 63, 38 to 63, 39 to 63, 40 to 63, 41 to 63, 42 to 63, 43 to 63, 44 to 63, 45 to 63, 46 to 63, 47 to 63, 48 to 63, 49 to 63, 50 to 63, 51 to 63, 52 to 63, 53 to 63, 54 to 63, 55 to 63, 56 to 63, 57 to 63, 58 to 63, 59 to 63, 60 to 63, 61 to 63, 62 to 63, 0 to 62, 1 to 62, 2 to 62, 3 to 62, 4 to 62, 5 to 62, 6 to 62, 7 to 62, 8 to 62, 9 to 62, 10 to 62, 11 to 62, 12 to 62, 13 to 62, 14 to 62, 15 to 62, 16 to 62, 17 to 62, 18 to 62, 19 to 62, 20 to 62, 21 to 62, 22 to 62, 23 to 62, 24 to 62, 25 to 62, 26 to 62, 27 to 62, 28 to 62, 29 to 62, 30 to 62, 31 to 62, 32 to 62, 33 to 62, 34 to 62, 35 to 62, 36 to 62, 37 to 62, 38 to 62, 39 to 62, 40 to 62, 41 to 62, 42 to 62, 43 to 62, 44 to 62, 45 to 62, 46 to 62, 47 to 62, 48 to 62, 49 to 62, 50 to 62, 51 to 62, 52 to 62, 53 to 62, 54 to 62, 55 to 62, 56 to 62, 57 to 62, 58 to 62, 59 to 62, 60 to 62, 61 to 62, 0 to 61, 1 to 61, 2 to 61, 3 to 61, 4 to 61, 5 to 61, 6 to 61, 7 to 61, 8 to 61, 9 to 61, 10 to 61, 11 to 61, 12 to 61, 13 to 61, 14 to 61, 15 to 61, 16 to 61, 17 to 61, 18 to 61, 19 to 61, 20 to 61, 21 to 61, 22 to 61, 23 to 61, 24 to 61, 25 to 61, 26 to 61, 27 to 61, 28 to 61, 29 to 61, 30 to 61, 31 to 61, 32 to 61, 33 to 61, 34 to 61, 35 to 61, 36 to 61, 37 to 61, 38 to 61, 39 to 61, 40 to 61, 41 to 61, 42 to 61, 43 to 61, 44 to 61, 45 to 61, 46 to 61, 47 to 61, 48 to 61, 49 to 61, 50 to 61, 51 to 61, 52 to 61, 53 to 61, 54 to 61, 55 to 61, 56 to 61, 57 to 61, 58 to 61, 59 to 61, 60 to 61, 0 to 60, 1 to 60, 2 to 60, 3 to 60, 4 to 60, 5 to 60, 6 to 60, 7 to 60, 8 to 60, 9 to 60, 10 to 60, 11 to 60, 12 to 60, 13 to 60, 14 to 60, 15 to 60, 16 to 60, 17 to 60, 18 to 60, 19 to 60, 20 to 60, 21 to 60, 22 to 60, 23 to 60, 24 to 60, 25 to 60, 26 to 60, 27 to 60, 28 to 60, 29 to 60, 30 to 60, 31 to 60, 32 to 60, 33 to 60, 34 to 60, 35 to 60, 36 to 60, 37 to 60, 38 to 60, 39 to 60, 40 to 60, 41 to 60, 42 to 60, 43 to 60, 44 to 60, 45 to 60, 46 to 60, 47 to 60, 48 to 60, 49 to 60, 50 to 60, 51 to 60, 52 to 60, 53 to 60, 54 to 60, 55 to 60, 56 to 60, 57 to 60, 58 to 60, 59 to 60, 0 to 59, 1 to 59, 2 to 59, 3 to 59, 4 to 59, 5 to 59, 6 to 59, 7 to 59, 8 to 59, 9 to 59, 10 to 59, 11 to 59, 12 to 59, 13 to 59, 14 to 59, 15 to 59, 16 to 59, 17 to 59, 18 to 59, 19 to 59, 20 to 59, 21 to 59, 22 to 59, 23 to 59, 24 to 59, 25 to 59, 26 to 59, 27 to 59, 28 to 59, 29 to 59, 30 to 59, 31 to 59, 32 to 59, 33 to 59, 34 to 59, 35 to 59, 36 to 59, 37 to 59, 38 to 59, 39 to 59, 40 to 59, 41 to 59, 42 to 59, 43 to 59, 44 to 59, 45 to 59, 46 to 59, 47 to 59, 48 to 59, 49 to 59, 50 to 59, 51 to 59, 52 to 59, 53 to 59, 54 to 59, 55 to 59, 56 to 59, 57 to 59, 58 to 59, 0 to 58, 1 to 58, 2 to 58, 3 to 58, 4 to 58, 5 to 58, 6 to 58, 7 to 58, 8 to 58, 9 to 58, 10 to 58, 11 to 58, 12 to 58, 13 to 58, 14 to 58, 15 to 58, 16 to 58, 17 to 58, 18 to 58, 19 to 58, 20 to 58, 21 to 58, 22 to 58, 23 to 58, 24 to 58, 25 to 58, 26 to 58, 27 to 58, 28 to 58, 29 to 58, 30 to 58, 31 to 58, 32 to 58, 33 to 58, 34 to 58, 35 to 58, 36 to 58, 37 to 58, 38 to 58, 39 to 58, 40 to 58, 41 to 58, 42 to 58, 43 to 58, 44 to 58, 45 to 58, 46 to 58, 47 to 58, 48 to 58, 49 to 58, 50 to 58, 51 to 58, 52 to 58, 53 to 58, 54 to 58, 55 to 58, 56 to 58, 57 to 58, 0 to 57, 1 to 57, 2 to 57, 3 to 57, 4 to 57, 5 to 57, 6 to 57, 7 to 57, 8 to 57, 9 to 57, 10 to 57, 11 to 57, 12 to 57, 13 to 57, 14 to 57, 15 to 57, 16 to 57, 17 to 57, 18 to 57, 19 to 57, 20 to 57, 21 to 57, 22 to 57, 23 to 57, 24 to 57, 25 to 57, 26 to 57, 27 to 57, 28 to 57, 29 to 57, 30 to 57, 31 to 57, 32 to 57, 33 to 57, 34 to 57, 35 to 57, 36 to 57, 37 to 57, 38 to 57, 39 to 57, 40 to 57, 41 to 57, 42 to 57, 43 to 57, 44 to 57, 45 to 57, 46 to 57, 47 to 57, 48 to 57, 49 to 57, 50 to 57, 51 to 57, 52 to 57, 53 to 57, 54 to 57, 55 to 57, 56 to 57, 0 to 56, 1 to 56, 2 to 56, 3 to 56, 4 to 56, 5 to 56, 6 to 56, 7 to 56, 8 to 56, 9 to 56, 10 to 56, 11 to 56, 12 to 56, 13 to 56, 14 to 56, 15 to 56, 16 to 56, 17 to 56, 18 to 56, 19 to 56, 20 to 56, 21 to 56, 22 to 56, 23 to 56, 24 to 56, 25 to 56, 26 to 56, 27 to 56, 28 to 56, 29 to 56, 30 to 56, 31 to 56, 32 to 56, 33 to 56, 34 to 56, 35 to 56, 36 to 56, 37 to 56, 38 to 56, 39 to 56, 40 to 56, 41 to 56, 42 to 56, 43 to 56, 44 to 56, 45 to 56, 46 to 56, 47 to 56, 48 to 56, 49 to 56, 50 to 56, 51 to 56, 52 to 56, 53 to 56, 54 to 56, 55 to 56, 0 to 55, 1 to 55, 2 to 55, 3 to 55, 4 to 55, 5 to 55, 6 to 55, 7 to 55, 8 to 55, 9 to 55, 10 to 55, 11 to 55, 12 to 55, 13 to 55, 14 to 55, 15 to 55, 16 to 55, 17 to 55, 18 to 55, 19 to 55, 20 to 55, 21 to 55, 22 to 55, 23 to 55, 24 to 55, 25 to 55, 26 to 55, 27 to 55, 28 to 55, 29 to 55, 30 to 55, 31 to 55, 32 to 55, 33 to 55, 34 to 55, 35 to 55, 36 to 55, 37 to 55, 38 to 55, 39 to 55, 40 to 55, 41 to 55, 42 to 55, 43 to 55, 44 to 55, 45 to 55, 46 to 55, 47 to 55, 48 to 55, 49 to 55, 50 to 55, 51 to 55, 52 to 55, 53 to 55, 54 to 55, 0 to 54, 1 to 54, 2 to 54, 3 to 54, 4 to 54, 5 to 54, 6 to 54, 7 to 54, 8 to 54, 9 to 54, 10 to 54, 11 to 54, 12 to 54, 13 to 54, 14 to 54, 15 to 54, 16 to 54, 17 to 54, 18 to 54, 19 to 54, 20 to 54, 21 to 54, 22 to 54, 23 to 54, 24 to 54, 25 to 54, 26 to 54, 27 to 54, 28 to 54, 29 to 54, 30 to 54, 31 to 54, 32 to 54, 33 to 54, 34 to 54, 35 to 54, 36 to 54, 37 to 54, 38 to 54, 39 to 54, 40 to 54, 41 to 54, 42 to 54, 43 to 54, 44 to 54, 45 to 54, 46 to 54, 47 to 54, 48 to 54, 49 to 54, 50 to 54, 51 to 54, 52 to 54, 53 to 54, 0 to 53, 1 to 53, 2 to 53, 3 to 53, 4 to 53, 5 to 53, 6 to 53, 7 to 53, 8 to 53, 9 to 53, 10 to 53, 11 to 53, 12 to 53, 13 to 53, 14 to 53, 15 to 53, 16 to 53, 17 to 53, 18 to 53, 19 to 53, 20 to 53, 21 to 53, 22 to 53, 23 to 53, 24 to 53, 25 to 53, 26 to 53, 27 to 53, 28 to 53, 29 to 53, 30 to 53, 31 to 53, 32 to 53, 33 to 53, 34 to 53, 35 to 53, 36 to 53, 37 to 53, 38 to 53, 39 to 53, 40 to 53, 41 to 53, 42 to 53, 43 to 53, 44 to 53, 45 to 53, 46 to 53, 47 to 53, 48 to 53, 49 to 53, 50 to 53, 51 to 53, 52 to 53, 0 to 52, 1 to 52, 2 to 52, 3 to 52, 4 to 52, 5 to 52, 6 to 52, 7 to 52, 8 to 52, 9 to 52, 10 to 52, 11 to 52, 12 to 52, 13 to 52, 14 to 52, 15 to 52, 16 to 52, 17 to 52, 18 to 52, 19 to 52, 20 to 52, 21 to 52, 22 to 52, 23 to 52, 24 to 52, 25 to 52, 26 to 52, 27 to 52, 28 to 52, 29 to 52, 30 to 52, 31 to 52, 32 to 52, 33 to 52, 34 to 52, 35 to 52, 36 to 52, 37 to 52, 38 to 52, 39 to 52, 40 to 52, 41 to 52, 42 to 52, 43 to 52, 44 to 52, 45 to 52, 46 to 52, 47 to 52, 48 to 52, 49 to 52, 50 to 52, 51 to 52, 0 to 51, 1 to 51, 2 to 51, 3 to 51, 4 to 51, 5 to 51, 6 to 51, 7 to 51, 8 to 51, 9 to 51, 10 to 51, 11 to 51, 12 to 51, 13 to 51, 14 to 51, 15 to 51, 16 to 51, 17 to 51, 18 to 51, 19 to 51, 20 to 51, 21 to 51, 22 to 51, 23 to 51, 24 to 51, 25 to 51, 26 to 51, 27 to 51, 28 to 51, 29 to 51, 30 to 51, 31 to 51, 32 to 51, 33 to 51, 34 to 51, 35 to 51, 36 to 51, 37 to 51, 38 to 51, 39 to 51, 40 to 51, 41 to 51, 42 to 51, 43 to 51, 44 to 51, 45 to 51, 46 to 51, 47 to 51, 48 to 51, 49 to 51, 50 to 51, 0 to 50, 1 to 50, 2 to 50, 3 to 50, 4 to 50, 5 to 50, 6 to 50, 7 to 50, 8 to 50, 9 to 50, 10 to 50, 11 to 50, 12 to 50, 13 to 50, 14 to 50, 15 to 50, 16 to 50, 17 to 50, 18 to 50, 19 to 50, 20 to 50, 21 to 50, 22 to 50, 23 to 50, 24 to 50, 25 to 50, 26 to 50, 27 to 50, 28 to 50, 29 to 50, 30 to 50, 31 to 50, 32 to 50, 33 to 50, 34 to 50, 35 to 50, 36 to 50, 37 to 50, 38 to 50, 39 to 50, 40 to 50, 41 to 50, 42 to 50, 43 to 50, 44 to 50, 45 to 50, 46 to 50, 47 to 50, 48 to 50, 49 to 50, 0 to 49, 1 to 49, 2 to 49, 3 to 49, 4 to 49, 5 to 49, 6 to 49, 7 to 49, 8 to 49, 9 to 49, 10 to 49, 11 to 49, 12 to 49, 13 to 49, 14 to 49, 15 to 49, 16 to 49, 17 to 49, 18 to 49, 19 to 49, 20 to 49, 21 to 49, 22 to 49, 23 to 49, 24 to 49, 25 to 49, 26 to 49, 27 to 49, 28 to 49, 29 to 49, 30 to 49, 31 to 49, 32 to 49, 33 to 49, 34 to 49, 35 to 49, 36 to 49, 37 to 49, 38 to 49, 39 to 49, 40 to 49, 41 to 49, 42 to 49, 43 to 49, 44 to 49, 45 to 49, 46 to 49, 47 to 49, 48 to 49, 0 to 48, 1 to 48, 2 to 48, 3 to 48, 4 to 48, 5 to 48, 6 to 48, 7 to 48, 8 to 48, 9 to 48, 10 to 48, 11 to 48, 12 to 48, 13 to 48, 14 to 48, 15 to 48, 16 to 48, 17 to 48, 18 to 48, 19 to 48, 20 to 48, 21 to 48, 22 to 48, 23 to 48, 24 to 48, 25 to 48, 26 to 48, 27 to 48, 28 to 48, 29 to 48, 30 to 48, 31 to 48, 32 to 48, 33 to 48, 34 to 48, 35 to 48, 36 to 48, 37 to 48, 38 to 48, 39 to 48, 40 to 48, 41 to 48, 42 to 48, 43 to 48, 44 to 48, 45 to 48, 46 to 48, 47 to 48, 0 to 47, 1 to 47, 2 to 47, 3 to 47, 4 to 47, 5 to 47, 6 to 47, 7 to 47, 8 to 47, 9 to 47, 10 to 47, 11 to 47, 12 to 47, 13 to 47, 14 to 47, 15 to 47, 16 to 47, 17 to 47, 18 to 47, 19 to 47, 20 to 47, 21 to 47, 22 to 47, 23 to 47, 24 to 47, 25 to 47, 26 to 47, 27 to 47, 28 to 47, 29 to 47, 30 to 47, 31 to 47, 32 to 47, 33 to 47, 34 to 47, 35 to 47, 36 to 47, 37 to 47, 38 to 47, 39 to 47, 40 to 47, 41 to 47, 42 to 47, 43 to 47, 44 to 47, 45 to 47, 46 to 47, 0 to 46, 1 to 46, 2 to 46, 3 to 46, 4 to 46, 5 to 46, 6 to 46, 7 to 46, 8 to 46, 9 to 46, 10 to 46, 11 to 46, 12 to 46, 13 to 46, 14 to 46, 15 to 46, 16 to 46, 17 to 46, 18 to 46, 19 to 46, 20 to 46, 21 to 46, 22 to 46, 23 to 46, 24 to 46, 25 to 46, 26 to 46, 27 to 46, 28 to 46, 29 to 46, 30 to 46, 31 to 46, 32 to 46, 33 to 46, 34 to 46, 35 to 46, 36 to 46, 37 to 46, 38 to 46, 39 to 46, 40 to 46, 41 to 46, 42 to 46, 43 to 46, 44 to 46, 45 to 46, 0 to 45, 1 to 45, 2 to 45, 3 to 45, 4 to 45, 5 to 45, 6 to 45, 7 to 45, 8 to 45, 9 to 45, 10 to 45, 11 to 45, 12 to 45, 13 to 45, 14 to 45, 15 to 45, 16 to 45, 17 to 45, 18 to 45, 19 to 45, 20 to 45, 21 to 45, 22 to 45, 23 to 45, 24 to 45, 25 to 45, 26 to 45, 27 to 45, 28 to 45, 29 to 45, 30 to 45, 31 to 45, 32 to 45, 33 to 45, 34 to 45, 35 to 45, 36 to 45, 37 to 45, 38 to 45, 39 to 45, 40 to 45, 41 to 45, 42 to 45, 43 to 45, 44 to 45, 0 to 44, 1 to 44, 2 to 44, 3 to 44, 4 to 44, 5 to 44, 6 to 44, 7 to 44, 8 to 44, 9 to 44, 10 to 44, 11 to 44, 12 to 44, 13 to 44, 14 to 44, 15 to 44, 16 to 44, 17 to 44, 18 to 44, 19 to 44, 20 to 44, 21 to 44, 22 to 44, 23 to 44, 24 to 44, 25 to 44, 26 to 44, 27 to 44, 28 to 44, 29 to 44, 30 to 44, 31 to 44, 32 to 44, 33 to 44, 34 to 44, 35 to 44, 36 to 44, 37 to 44, 38 to 44, 39 to 44, 40 to 44, 41 to 44, 42 to 44, 43 to 44, 0 to 43, 1 to 43, 2 to 43, 3 to 43, 4 to 43, 5 to 43, 6 to 43, 7 to 43, 8 to 43, 9 to 43, 10 to 43, 11 to 43, 12 to 43, 13 to 43, 14 to 43, 15 to 43, 16 to 43, 17 to 43, 18 to 43, 19 to 43, 20 to 43, 21 to 43, 22 to 43, 23 to 43, 24 to 43, 25 to 43, 26 to 43, 27 to 43, 28 to 43, 29 to 43, 30 to 43, 31 to 43, 32 to 43, 33 to 43, 34 to 43, 35 to 43, 36 to 43, 37 to 43, 38 to 43, 39 to 43, 40 to 43, 41 to 43, 42 to 43, 0 to 42, 1 to 42, 2 to 42, 3 to 42, 4 to 42, 5 to 42, 6 to 42, 7 to 42, 8 to 42, 9 to 42, 10 to 42, 11 to 42, 12 to 42, 13 to 42, 14 to 42, 15 to 42, 16 to 42, 17 to 42, 18 to 42, 19 to 42, 20 to 42, 21 to 42, 22 to 42, 23 to 42, 24 to 42, 25 to 42, 26 to 42, 27 to 42, 28 to 42, 29 to 42, 30 to 42, 31 to 42, 32 to 42, 33 to 42, 34 to 42, 35 to 42, 36 to 42, 37 to 42, 38 to 42, 39 to 42, 40 to 42, 41 to 42, 0 to 41, 1 to 41, 2 to 41, 3 to 41, 4 to 41, 5 to 41, 6 to 41, 7 to 41, 8 to 41, 9 to 41, 10 to 41, 11 to 41, 12 to 41, 13 to 41, 14 to 41, 15 to 41, 16 to 41, 17 to 41, 18 to 41, 19 to 41, 20 to 41, 21 to 41, 22 to 41, 23 to 41, 24 to 41, 25 to 41, 26 to 41, 27 to 41, 28 to 41, 29 to 41, 30 to 41, 31 to 41, 32 to 41, 33 to 41, 34 to 41, 35 to 41, 36 to 41, 37 to 41, 38 to 41, 39 to 41, 40 to 41, 0 to 40, 1 to 40, 2 to 40, 3 to 40, 4 to 40, 5 to 40, 6 to 40, 7 to 40, 8 to 40, 9 to 40, 10 to 40, 11 to 40, 12 to 40, 13 to 40, 14 to 40, 15 to 40, 16 to 40, 17 to 40, 18 to 40, 19 to 40, 20 to 40, 21 to 40, 22 to 40, 23 to 40, 24 to 40, 25 to 40, 26 to 40, 27 to 40, 28 to 40, 29 to 40, 30 to 40, 31 to 40, 32 to 40, 33 to 40, 34 to 40, 35 to 40, 36 to 40, 37 to 40, 38 to 40, 39 to 40, 0 to 39, 1 to 39, 2 to 39, 3 to 39, 4 to 39, 5 to 39, 6 to 39, 7 to 39, 8 to 39, 9 to 39, 10 to 39, 11 to 39, 12 to 39, 13 to 39, 14 to 39, 15 to 39, 16 to 39, 17 to 39, 18 to 39, 19 to 39, 20 to 39, 21 to 39, 22 to 39, 23 to 39, 24 to 39, 25 to 39, 26 to 39, 27 to 39, 28 to 39, 29 to 39, 30 to 39, 31 to 39, 32 to 39, 33 to 39, 34 to 39, 35 to 39, 36 to 39, 37 to 39, 38 to 39, 0 to 38, 1 to 38, 2 to 38, 3 to 38, 4 to 38, 5 to 38, 6 to 38, 7 to 38, 8 to 38, 9 to 38, 10 to 38, 11 to 38, 12 to 38, 13 to 38, 14 to 38, 15 to 38, 16 to 38, 17 to 38, 18 to 38, 19 to 38, 20 to 38, 21 to 38, 22 to 38, 23 to 38, 24 to 38, 25 to 38, 26 to 38, 27 to 38, 28 to 38, 29 to 38, 30 to 38, 31 to 38, 32 to 38, 33 to 38, 34 to 38, 35 to 38, 36 to 38, 37 to 38, 0 to 37, 1 to 37, 2 to 37, 3 to 37, 4 to 37, 5 to 37, 6 to 37, 7 to 37, 8 to 37, 9 to 37, 10 to 37, 11 to 37, 12 to 37, 13 to 37, 14 to 37, 15 to 37, 16 to 37, 17 to 37, 18 to 37, 19 to 37, 20 to 37, 21 to 37, 22 to 37, 23 to 37, 24 to 37, 25 to 37, 26 to 37, 27 to 37, 28 to 37, 29 to 37, 30 to 37, 31 to 37, 32 to 37, 33 to 37, 34 to 37, 35 to 37, 36 to 37, 0 to 36, 1 to 36, 2 to 36, 3 to 36, 4 to 36, 5 to 36, 6 to 36, 7 to 36, 8 to 36, 9 to 36, 10 to 36, 11 to 36, 12 to 36, 13 to 36, 14 to 36, 15 to 36, 16 to 36, 17 to 36, 18 to 36, 19 to 36, 20 to 36, 21 to 36, 22 to 36, 23 to 36, 24 to 36, 25 to 36, 26 to 36, 27 to 36, 28 to 36, 29 to 36, 30 to 36, 31 to 36, 32 to 36, 33 to 36, 34 to 36, 35 to 36, 0 to 35, 1 to 35, 2 to 35, 3 to 35, 4 to 35, 5 to 35, 6 to 35, 7 to 35, 8 to 35, 9 to 35, 10 to 35, 11 to 35, 12 to 35, 13 to 35, 14 to 35, 15 to 35, 16 to 35, 17 to 35, 18 to 35, 19 to 35, 20 to 35, 21 to 35, 22 to 35, 23 to 35, 24 to 35, 25 to 35, 26 to 35, 27 to 35, 28 to 35, 29 to 35, 30 to 35, 31 to 35, 32 to 35, 33 to 35, 34 to 35, 0 to 34, 1 to 34, 2 to 34, 3 to 34, 4 to 34, 5 to 34, 6 to 34, 7 to 34, 8 to 34, 9 to 34, 10 to 34, 11 to 34, 12 to 34, 13 to 34, 14 to 34, 15 to 34, 16 to 34, 17 to 34, 18 to 34, 19 to 34, 20 to 34, 21 to 34, 22 to 34, 23 to 34, 24 to 34, 25 to 34, 26 to 34, 27 to 34, 28 to 34, 29 to 34, 30 to 34, 31 to 34, 32 to 34, 33 to 34, 0 to 33, 1 to 33, 2 to 33, 3 to 33, 4 to 33, 5 to 33, 6 to 33, 7 to 33, 8 to 33, 9 to 33, 10 to 33, 11 to 33, 12 to 33, 13 to 33, 14 to 33, 15 to 33, 16 to 33, 17 to 33, 18 to 33, 19 to 33, 20 to 33, 21 to 33, 22 to 33, 23 to 33, 24 to 33, 25 to 33, 26 to 33, 27 to 33, 28 to 33, 29 to 33, 30 to 33, 31 to 33, 32 to 33, 0 to 32, 1 to 32, 2 to 32, 3 to 32, 4 to 32, 5 to 32, 6 to 32, 7 to 32, 8 to 32, 9 to 32, 10 to 32, 11 to 32, 12 to 32, 13 to 32, 14 to 32, 15 to 32, 16 to 32, 17 to 32, 18 to 32, 19 to 32, 20 to 32, 21 to 32, 22 to 32, 23 to 32, 24 to 32, 25 to 32, 26 to 32, 27 to 32, 28 to 32, 29 to 32, 30 to 32, 31 to 32, 0 to 31, 1 to 31, 2 to 31, 3 to 31, 4 to 31, 5 to 31, 6 to 31, 7 to 31, 8 to 31, 9 to 31, 10 to 31, 11 to 31, 12 to 31, 13 to 31, 14 to 31, 15 to 31, 16 to 31, 17 to 31, 18 to 31, 19 to 31, 20 to 31, 21 to 31, 22 to 31, 23 to 31, 24 to 31, 25 to 31, 26 to 31, 27 to 31, 28 to 31, 29 to 31, 30 to 31, 0 to 30, 1 to 30, 2 to 30, 3 to 30, 4 to 30, 5 to 30, 6 to 30, 7 to 30, 8 to 30, 9 to 30, 10 to 30, 11 to 30, 12 to 30, 13 to 30, 14 to 30, 15 to 30, 16 to 30, 17 to 30, 18 to 30, 19 to 30, 20 to 30, 21 to 30, 22 to 30, 23 to 30, 24 to 30, 25 to 30, 26 to 30, 27 to 30, 28 to 30, 29 to 30, 0 to 29, 1 to 29, 2 to 29, 3 to 29, 4 to 29, 5 to 29, 6 to 29, 7 to 29, 8 to 29, 9 to 29, 10 to 29, 11 to 29, 12 to 29, 13 to 29, 14 to 29, 15 to 29, 16 to 29, 17 to 29, 18 to 29, 19 to 29, 20 to 29, 21 to 29, 22 to 29, 23 to 29, 24 to 29, 25 to 29, 26 to 29, 27 to 29, 28 to 29, 0 to 28, 1 to 28, 2 to 28, 3 to 28, 4 to 28, 5 to 28, 6 to 28, 7 to 28, 8 to 28, 9 to 28, 10 to 28, 11 to 28, 12 to 28, 13 to 28, 14 to 28, 15 to 28, 16 to 28, 17 to 28, 18 to 28, 19 to 28, 20 to 28, 21 to 28, 22 to 28, 23 to 28, 24 to 28, 25 to 28, 26 to 28, 27 to 28, 0 to 27, 1 to 27, 2 to 27, 3 to 27, 4 to 27, 5 to 27, 6 to 27, 7 to 27, 8 to 27, 9 to 27, 10 to 27, 11 to 27, 12 to 27, 13 to 27, 14 to 27, 15 to 27, 16 to 27, 17 to 27, 18 to 27, 19 to 27, 20 to 27, 21 to 27, 22 to 27, 23 to 27, 24 to 27, 25 to 27, 26 to 27, 0 to 26, 1 to 26, 2 to 26, 3 to 26, 4 to 26, 5 to 26, 6 to 26, 7 to 26, 8 to 26, 9 to 26, 10 to 26, 11 to 26, 12 to 26, 13 to 26, 14 to 26, 15 to 26, 16 to 26, 17 to 26, 18 to 26, 19 to 26, 20 to 26, 21 to 26, 22 to 26, 23 to 26, 24 to 26, 25 to 26, 0 to 25, 1 to 25, 2 to 25, 3 to 25, 4 to 25, 5 to 25, 6 to 25, 7 to 25, 8 to 25, 9 to 25, 10 to 25, 11 to 25, 12 to 25, 13 to 25, 14 to 25, 15 to 25, 16 to 25, 17 to 25, 18 to 25, 19 to 25, 20 to 25, 21 to 25, 22 to 25, 23 to 25, 24 to 25, 0 to 24, 1 to 24, 2 to 24, 3 to 24, 4 to 24, 5 to 24, 6 to 24, 7 to 24, 8 to 24, 9 to 24, 10 to 24, 11 to 24, 12 to 24, 13 to 24, 14 to 24, 15 to 24, 16 to 24, 17 to 24, 18 to 24, 19 to 24, 20 to 24, 21 to 24, 22 to 24, 23 to 24, 0 to 23, 1 to 23, 2 to 23, 3 to 23, 4 to 23, 5 to 23, 6 to 23, 7 to 23, 8 to 23, 9 to 23, 10 to 23, 11 to 23, 12 to 23, 13 to 23, 14 to 23, 15 to 23, 16 to 23, 17 to 23, 18 to 23, 19 to 23, 20 to 23, 21 to 23, 22 to 23, 0 to 22, 1 to 22, 2 to 22, 3 to 22, 4 to 22, 5 to 22, 6 to 22, 7 to 22, 8 to 22, 9 to 22, 10 to 22, 11 to 22, 12 to 22, 13 to 22, 14 to 22, 15 to 22, 16 to 22, 17 to 22, 18 to 22, 19 to 22, 20 to 22, 21 to 22, 0 to 21, 1 to 21, 2 to 21, 3 to 21, 4 to 21, 5 to 21, 6 to 21, 7 to 21, 8 to 21, 9 to 21, 10 to 21, 11 to 21, 12 to 21, 13 to 21, 14 to 21, 15 to 21, 16 to 21, 17 to 21, 18 to 21, 19 to 21, 20 to 21, 0 to 20, 1 to 20, 2 to 20, 3 to 20, 4 to 20, 5 to 20, 6 to 20, 7 to 20, 8 to 20, 9 to 20, 10 to 20, 11 to 20, 12 to 20, 13 to 20, 14 to 20, 15 to 20, 16 to 20, 17 to 20, 18 to 20, 19 to 20, 0 to 19, 1 to 19, 2 to 19, 3 to 19, 4 to 19, 5 to 19, 6 to 19, 7 to 19, 8 to 19, 9 to 19, 10 to 19, 11 to 19, 12 to 19, 13 to 19, 14 to 19, 15 to 19, 16 to 19, 17 to 19, 18 to 19, 0 to 18, 1 to 18, 2 to 18, 3 to 18, 4 to 18, 5 to 18, 6 to 18, 7 to 18, 8 to 18, 9 to 18, 10 to 18, 11 to 18, 12 to 18, 13 to 18, 14 to 18, 15 to 18, 16 to 18, 17 to 18, 0 to 17, 1 to 17, 2 to 17, 3 to 17, 4 to 17, 5 to 17, 6 to 17, 7 to 17, 8 to 17, 9 to 17, 10 to 17, 11 to 17, 12 to 17, 13 to 17, 14 to 17, 15 to 17, 16 to 17, 0 to 16, 1 to 16, 2 to 16, 3 to 16, 4 to 16, 5 to 16, 6 to 16, 7 to 16, 8 to 16, 9 to 16, 10 to 16, 11 to 16, 12 to 16, 13 to 16, 14 to 16, 15 to 16, 0 to 15, 1 to 15, 2 to 15, 3 to 15, 4 to 15, 5 to 15, 6 to 15, 7 to 15, 8 to 15, 9 to 15, 10 to 15, 11 to 15, 12 to 15, 13 to 15, 14 to 15, 0 to 14, 1 to 14, 2 to 14, 3 to 14, 4 to 14, 5 to 14, 6 to 14, 7 to 14, 8 to 14, 9 to 14, 10 to 14, 11 to 14, 12 to 14, 13 to 14, 0 to 13, 1 to 13, 2 to 13, 3 to 13, 4 to 13, 5 to 13, 6 to 13, 7 to 13, 8 to 13, 9 to 13, 10 to 13, 11 to 13, 12 to 13, 0 to 12, 1 to 12, 2 to 12, 3 to 12, 4 to 12, 5 to 12, 6 to 12, 7 to 12, 8 to 12, 9 to 12, 10 to 12, 11 to 12, 0 to 11, 1 to 11, 2 to 11, 3 to 11, 4 to 11, 5 to 11, 6 to 11, 7 to 11, 8 to 11, 9 to 11, 10 to 11, 0 to 10, 1 to 10, 2 to 10, 3 to 10, 4 to 10, 5 to 10, 6 to 10, 7 to 10, 8 to 10, 9 to 10, 0 to 9, 1 to 9, 2 to 9, 3 to 9, 4 to 9, 5 to 9, 6 to 9, 7 to 9, 8 to 9, 0 to 8, 1 to 8, 2 to 8, 3 to 8, 4 to 8, 5 to 8, 6 to 8, 7 to 8, 0 to 7, 1 to 7, 2 to 7, 3 to 7, 4 to 7, 5 to 7, 6 to 7, 0 to 6, 1 to 6, 2 to 6, 3 to 6, 4 to 6, 5 to 6, 0 to 5, 1 to 5, 2 to 5, 3 to 5, 4 to 5, 0 to 4, 1 to 4, 2 to 4, 3 to 4, 0 to 3, 1 to 3, 2 to 3, 0 to 2, 1 to 2, or 0 to 1 weight % water.

The acetyl feed stream 15 can be mixed with liquid or vaporous water, i.e., steam, so that the feed stream comprises about 40 to about 99 weight % acetic acid, up to about 50 weight of the impurity, and optionally up to about 30 weight % water, based on the total weight of the feed stream 15.

The acetyl feed stream 15 may contain up to about 10 weight % acetic anhydride, or about 0.5 to about 10 weight % acetic anhydride, or about 1.0 to about 10 weight % acetic anhydride, or about 2.0 to about 10 weight % acetic anhydride, or about 3.0 to about 10 weight % acetic anhydride, or about 4.0 to about 10 weight % acetic anhydride, or about 5.0 to about 10 weight % acetic anhydride, or about 6.0 to about 10 weight % acetic anhydride, or about 7.0 to about 10 weight % acetic anhydride, or about 8.0 to about 10 weight % acetic anhydride. It is understood that such ranges include 0.5 to 9, 1 to 9, 2 to 9, 3 to 9, 4 to 9, 5 to 9, 6 to 9, 7 to 9, 8 to 9, 0.5 to 8, 1 to 8, 2 to 8, 3 to 8, 4 to 8, 5 to 8, 6 to 8, 7 to 8, 0.5 to 7, 1 to 7, 2 to 7, 3 to 7, 4 to 7, 5 to 7, 6 to 7, 0.5 to 6, 1 to 6, 2 to 6, 3 to 6, 4 to 6, 5 to 6, 0.5 to 5, 1 to 5, 2 to 5, 3 to 5, 4 to 5, 0.5 to 4, 1 to 4, 2 to 4, 3 to 4, 0.5 to 3, 1 to 3, 2 to 3, 0.5 to 2, 1 to 2, and 0.5 to 1 weight % acetic anhydride wherein the weight percentage is based on the total constituents of the feed stream 15.

Optionally, acetic acid may also be added to the wet acetic acid feed stream to bring the final concentration of acetic acid to one of the aforementioned ranges. It is to be understood that the above delineated weight percentages are based on the total weight of all the constituents present in the acetyl feed stream 15. It is also to be understood that the ranges specified include all concentrations, weight percentages and ranges in between the ranges specified and that such ranges have been specified as whole numbers for sake of brevity.

In the vaporization unit 20 typically 75 to 99%, of the acetyl feed 15 is vaporized by boiling against steam, to produce vaporized acid stream 25. The acid feed stream 15 is vaporized at typically 110° C.-195° C. and at a pressure of from 0.7 to 7.0 bar, or from about 115° C.-160° C., and at a pressure of from 0.9 to 3.2 bar. Typically, 1.0 weight % to 25.0 weight % of the incoming feed acid stream 15 may be removed as sludge by stream 30 from the vaporizer 20 to prevent fouling of the vaporizer equipment, the furnace superheater 45, and the catalyst bed, as well as to remove non-volatile components such as salts and tars. The vaporizer 20 can be any apparatus known to persons skilled in the art such as, for example, kettle-type, thermosyphon-type, wiped-film, falling film, and thin film evaporators. Optionally, steam stream 35 may be added to the vaporized acid to bring the water concentration in the acid stream 40 from about 5 weight % to about 70 weight % water, or the wet acid stream can have from about 10 weight % to about 20 weight % water, based on the total weight of the constituents in stream 40. This water addition helps mitigate coke formation in the ketonization reactor and increases the yield of acetone from acetic acid.

Wet acid stream 40 is further superheated to the desired reaction inlet temperature in a superheater or furnace 45 to produce superheated feed stream 50. The term “superheated,” as used herein, is intended to have the commonly understood meaning of a vapor heated to a temperature above its dew point at a given pressure. The temperature of the superheated feed stream 50 can be about 350° C. to about 650° C., or from about 350° C. to about 600° C., or from about 350° C. to about 550° C., or from about 300° C. to about 450° C. Typically, the wet acid feed stream 40 is preheated to a predetermined inlet temperature in a furnace and then passed through the ketonization catalyst bed.

The vaporized feed mixture 40 may be conveyed through the superheater 45 via a multi-pass tubular configuration inside of an insulated furnace box. If a direct fired furnace is used, then heat is provided to the furnace by combustion of fuel 52 with air stream 53, and diluted for temperature control by at least a portion of a by-product carbon dioxide stream 54 via conduit 55. The fuel for the furnace may be any combustible material of sufficient energy density, including, but not limited to natural gas, propane, butane, natural gas liquids, liquefied petroleum gases, hydrogen, refinery off gases, pyrolysis gasoline, ethanol, methanol, heavy organic by-products from the ketonization reactor, such as mesityl oxide and related compounds, the sludge stream 30 from the acid vaporizer 20, or petroleum fractions, such as gasoline, kerosene, bunker fuel, heating oil, and the like. Design of the burners is highly dependent on the fuel chosen as is well known to those skilled in the art. Natural gas is the preferred furnace fuel.

Heat may be transferred to the tubes containing the wet acid feed 40 via radiated and convective heat transfer mechanisms. In order to prevent high tube skin temperatures which tend to lead to coking on the process fluid side of the tubes, it is desirable that the primary heat transfer occur in a portion of the feed superheater 45 where sufficient diluent has been added to the already combusted hot fuel/air mixture to lower its temperature to 700° C. to 900° C. in a post combustion zone. The diluent gas may be air or by-product carbon dixode stream or a combinations thereof. The preferred diluent, above the excess air required for combustion, is the by-product carbon dioxide stream 54. Although any conventional source of oxygen can be used, air is generally the least expensive and most readily available source of oxygen. Such furnace configurations are described in greater detail in U.S. Pat. No. 8,779,208, the entire disclosure of which is incorporated herein by reference.

Air feed to the combustion zone of the furnace 45 may be via natural or forced draft. Sufficient air is supplied to give 10 to 40% excess oxygen over the stoichiometric amount required for complete combustion of both the fuel and the VOC components in the by-product carbon dioxide stream. If the by-product carbon dioxide stream is utilized for combustion, then, desirably, residence time in the post combustion zone for the oxidative destruction of the VOC's can be from 0.02 to 5.0 seconds, or from 0.1 to 0.5 seconds. The temperature in the post combustion zone of the furnace where VOC destruction takes place can be from 600° C.-900° C., or from 650° C.-800° C. The furnace is designed such that residence time and temperature are sufficient for at least 50 mole %, or at least 65 mole % of the total VOC's present originally in the by-product carbon dioxide stream are oxidatively destroyed.

The superheater furnace 45 is sized to supply sufficient heat to raise the wet acid feed 50 to proper reaction temperature, providing both sensible heat and sufficient thermal energy to compensate for the endothermic heat of ketonization. Typically the furnace will be designed to supply 0.7 to 2.6 million J/kg, more typically 0.75-0.9 million J/kg of acetic acid fed, depending on water content of the vaporized acid stream.

When run in adiabatic mode, the wet acid feed 15 is preheated to the desired reactor inlet temperature, typically 350° C. to 650° C., or it can be from 350° C. to 500° C. in a direct-fired furnace or superheater 45 in order to supply the heat of reaction. As discussed above, it is common for the acid feed 15 to be conveyed through the superheater 45 via a multi-pass tubular configuration situated in an insulated furnace box wherein a fuel is combusted with oxygen and diluent to generate high temperature heat.

The superheated feed stream 50 coming from the superheater 45 is passed to the ketonization reactor 60 where the acetic acid and other reactive feed molecules, if present, are converted over a heterogeneous ketonization catalyst to a crude product mixture comprising acetone, water, carbon dioxide, unreacted acetic acid, acetic acid azeotrope-forming compounds, and other minor by-products to produce crude product mixture 65. The ketonization reactor 60 can be any reactor format known in the art to be suitable for gas-phase endothermic reactions. For example, the ketonization reaction may be conducted using a fixed, fluidized, or moving bed reactor. The ketonization reaction can be carried out in a single stage adiabatic fixed bed reactor; a multiple-stage adiabatic fixed bed reactor with interstage heating or hot-shotting; or a tubular fixed bed reactor in a fired furnace or molten salt heating bath.

Typically, about 90 mole % to about 100 mole % of the acetic acid will be converted to acetone, carbon dioxide, and water in a single stage adiabatic reactor. The inlet pressure to the ketonization reactor can be from about 0.5 bars to about 10 bars absolute. The temperature range for the ketonization reactor can be about 300° C. to about 600° C. over the length of the reactor. Preferably, the reaction is carried out in the vapor phase at elevated temperatures under the following conditions. The reaction temperature may be at least 300° C., or at least 325° C., or at least 350° C. In terms of ranges, the reaction temperature may range from 300° C. to 550° C., or from 325° C. to 500° C., or from 350° C. to 450° C. The pressure may range from 0.5 bars to about 10 bars absolute, or from 0.5 bars to about 8 bars absolute, or from 0.9 to about 7 bars absolute, or from 1.1 to about 5 bars absolute. The reactants may be fed to the reactor 60 at a gas hourly space velocity (GHSV) greater than 500 hr.⁻¹, or greater than 1000 hr.⁻¹, or in greater than 2500 hr.⁻¹ or even greater than 5000 hr.⁻¹. In terms of ranges the GHSV may range from 50 hr.⁻¹ to 50,000 hr.⁻¹, or from 500 hr.⁻¹ to 30,000 hr.⁻¹, or from 1000 hr.⁻¹ to 10,000 hr.⁻¹, or from 1000 hr.⁻¹ to 6500 hr.⁻¹. When run in single stage, adiabatic mode, the reactor temperature will be highest at the inlet and drop to the lowest value at the outlet because of the endothermic heat of reaction. The temperature drop across the reactor can be as much as from about 40° to about 75° C., depending on water content of the feed and conversion of acetic acid.

Contact or residence time can vary widely, depending upon such variables as amount of acetic acid, catalyst, reactor, temperature, and pressure. Typical contact times range from a fraction of a second to more than several hours when a catalyst system other than a fixed bed is used, with preferred contact times from 0.1 to 100 seconds, or from 0.3 to 80 seconds, or from 0.4 to 30 seconds. One skilled in the art will understand that the contact or residence time can vary due to many factors present in ketonization reactor such as pressure, temperature, catalyst activity, catalyst selectivity, flow through, and the like. Accordingly, adjustment of the residence time to obtain the level of conversion of acetyl to ketone, such as acetic acid to acetone, is well within the understanding of one skilled in the art.

In the ketonization reactor, the superheated feed mixture 50 contacts a metal oxide catalyst where the acetic acid and other reactive species, such as trace amounts of propionic acid or acetic anhydride, are converted into a gaseous crude product mixture 65 comprising acetone, other ketones, water, the impurity having at least one acetic acid azeotrope-forming compound, and byproducts from the ketonization reaction. Such byproducts include, for example, carbon dioxide, and one or more volatile organic compounds having a boiling point less than or equal to 250° C. measured at a standard atmospheric pressure of 1 bar absolute. Some examples of volatile organic compounds include, but are not limited to, methane, ethane, acetone, methyl acetate, isobutylene, mesityl oxide, terpenes, methyl ethyl ketone, and other low molecular weight aldehydes, ketones, hydrocarbons, olefins, alcohols, and esters. The crude product mixture 65 comprises from 25 to about 70 weight % acetone, about 25 to about 75 weight % water, and about 10 ppm to about 25 weight % of the impurity, wherein the weight % is based on the total weight of the constituents of the product mixture 65 and absent any catalyst carryover; or from 40 to about 70 weight % acetone, about 30 to about 60 weight % water, and about 100 ppm to about 20 weight % of the impurity; or from 50 to about 70 weight % acetone, about 30 to about 35 weight % water, and about 200 ppm to about 15 weight % of the impurity.

Optionally, the vaporized acetic acid 50 may be fed to the ketonization reactor 60 along with a carrier gas. The acetic acid is transferred to the vapor state by passing a carrier gas through the acetic acid at a temperature at or below 150° C., followed by heating the gaseous stream to the reactor inlet temperature. In the case where a carrier gas is utilized, it may be selected from such gases as hydrogen, nitrogen, argon, helium, carbon dioxide or combinations thereof. Although the carrier gas may be inert, it is also contemplated that hydrogen can be used which may also reduce the acetic acid.

The ketonization reactor may be operated in isothermal mode. The ketonization catalyst is charged to tubes placed in a furnace box and reaction occurs simultaneously with direct-fired heating.

The metal oxide catalyst(s) utilized in the ketonization reaction of the present invention include oxides rare earth metals, transition metals, alkali metals, and alkaline earth metals, either alone or in combination with one or more metals. The metal oxide catalysts can exhibit both acid and base functionalities. The metal oxides may be employed either alone or in combination with one or more metals. Representative examples of metal oxide ketonization catalysts may be found in Glinski et al, “Ketones from Monocarboxylic Acids: Catalytic Ketonization Over Oxide Catalysts”, Applied Catalysis A: General, Vol. 128, (1995) pp. 209-217. The metal oxides may be supported on inorganic carriers well-known to persons skilled in the art such as, for example, silica, titania, or alumina. The activity and selectivity of the metal oxide catalyst may be enhanced by the presence of metal oxides of the Group IA metals, such as lithium, sodium, potassium, and cesium as disclosed, for example, by U.S. Pat. No. 4,950,763.

For producing acetone, the type of support influences the conversion of acetic acid and selectivity to acetone. Some specific examples of metal oxide ketonization catalysts include, but are not limited to, oxides of cerium, thorium, lanthanum, manganese, zirconium, titanium, zinc, chromium, lead, iron, niobium, molybdenum, bismuth, cadmium, copper, nickel, magnesium, aluminum, and mixtures thereof. For example, the superheated feed stream can have a temperature of about 300° C. to about 600° C., the metal oxide catalyst can comprise an oxide of titanium, zirconium, thorium, cerium, lanthanum, or a mixture thereof. The support can be present in an amount from 50 weight % to 99.5 weight %, or from 75 weight % to 99 weight %, or from 80 weight % to 90 weight %, based on the weight of the catalyst.

The metal oxide catalyst may be further impregnated with about 0.05 to about 50 weight %, or about 1 weight % to about 25 weight %, or about 10 weight % to about 20 weight %, based on the total weight of the catalyst, of lithium, sodium, potassium, cesium, lanthanum, cerium, or a combination thereof. Alternatively, the catalyst can be impregnated with about 0.05 to about 50 weight %, or about 1 weight % to about 25 weight %, or about 10 weight % to about 20 weight %, based on the total weight of the catalyst, of lithium, sodium, potassium, cesium, or a mixture thereof. It is also within the inventive concept for the ketonization catalyst to comprise titanium dioxide impregnated with about 1 to about 10 weight %, based on the weight of the catalyst of at least one of lithium, sodium, cesium, or potassium. The metal loading may vary depending on the type of active metal. The titanium dioxide can be in the anatase form.

The surface area of the ketonization catalyst can range from about 10 to about 400 m²/g of catalyst. Other examples of catalyst surface areas are about 20 to about 250 m²/g, or 50 to about 200 m²/g. The impregnated and/or supported catalysts can be prepared in accordance with methods well-known to persons skilled in the art such as, for example, by thoroughly mixing metal salt solutions of the catalyst and optional catalyst promoter with the carrier or support material. Capillary action then draws the precursor into the pores in the support. The catalyst is then dried and calcined. The catalyst may be in any of the commonly used catalyst shapes such as, for example, spheres, granules, pellets, chips, rings, extrudates, or powders that are well-known in the art. The ketonization catalyst can be regenerated by heating in the presence of an oxygen-containing gas at a temperature of about 375° C. to about 550° C.

The crude product mixture or gaseous reactor effluent 65, is cooled and separated in recovery zone 70 to produce a gaseous, non-condensable, by-product carbon dioxide stream 54 and a liquid crude acetone stream 75. The by-product carbon dioxide stream 54 comprises non-condensable compounds such as carbon dioxide, isobutylene, methane, hydrogen, other minor VOC's, and traces of acetone and higher by-products. The by-product carbon dioxide stream 54, may be sent in its entirety via conduit 55 to furnace 45, or a portion emitted directly via conduit 77 for proper disposal. Typically, during normal operation all of stream 54 will be sent to furnace 45 for combustion of VOC's, although at start up, or during furnace up-sets, a fraction or all of stream 54 may exit the process via stream 77 without further treatment. The liquid crude acetone stream 75 comprises the majority of the acetone, water, impurity and heavy by-products.

In the recovery zone 70 the ketone component can be separated from the carbon dioxide, carrier gas, if utilized, and one or more ketonization byproducts by conventional methods known to persons skilled in the art. For example, as illustrated by the ketonization of acetic acid to acetone, the gaseous product mixture from the ketonization reactor can be separated by direct condensation or absorption of the gaseous ketonization reactor product mixture into water or other solvent to produce a condensed crude acetone stream and a vaporous non-condensable byproduct stream comprising carbon dioxide and the byproducts such as isobutylene, hydrogen, methane, and higher ketones.

The separation step comprises cooling the gaseous product mixture by contact with a heat exchanger or a solvent. For example, the ketone component, i.e. acetone, may be condensed by indirect cooling in a heat exchanger against water, chilled brine, chilled glycol or the like, or via direct contact cooling with an injected solvent, such as water. After cooling, phase separation produces a vapor byproduct stream comprising the majority of the non-condensable components (such as, carbon dioxide, methane, isobutylene, and hydrogen), along with small amounts of acetone and higher boiling impurities; and a liquid acetone stream comprising the majority of the acetone, water, heavy byproducts from the reactor and the impurity having at least one acetic acid azeotrope-forming compound. The temperature range of the condenser operation can be from 0° C. to about 40° C., or from about 5° C. to 25° C. The condensed effluent from the ketonization reaction comprises about 25 to about 70 weight % acetone, about 25 to about 75 weight % water, and about 10 ppm to about 25 weight % of the acetic acid azeotrope-forming impurities and may further include about 0.1 to about 2 weight % mesityl oxide, wherein the weight % is based on the total weight of the constituents in the effluent. Generally recovery of acetone by condensation results in about 90 mole % recovery of the acetone, or greater than 95 mole % of the acetone is recovered, or greater than about 99 mole % of the acetone is recovered, based on the acetone fed to the condenser (recovery zone 70).

High recovery of acetone by condensation alone, however, requires very low temperatures because of the volatile nature of acetone and the large volume of the non-condensable carbon dioxide present in the gaseous reactor effluent. The invention also includes recovering the acetone from the gaseous reactor effluent by absorption into a solvent such as, for example, water. Generally, the recovery of acetone by countercurrent absorption into water results in about 99 mole %, or about 99.5 mole %, or about 99.8 mole % recovery of acetone, based on the acetone fed to the absorber. The absorption may be carried out by any means known to those skilled in the art, for example, by contacting the gaseous crude product mixture with water in a countercurrent absorber such as, for example, a packed or trayed absorption tower. The gaseous crude product mixture containing acetone can be fed to the bottom of the absorption tower and acetone-lean solvent, e.g., water, can be fed to the top of the tower, which permits the gas and liquid phases co-mingle in a countercurrent flow pattern. The gaseous crude absorber stream comprises a vaporous acetone-lean carbon dioxide stream that is removed from the top of the tower or absorber, and the liquid crude absorber product stream comprises an acetone-rich stream which is removed from the bottom of the column. The gaseous crude absorber stream comprises less than about 50 mole % of the acetone in the crude product mixture coming from the ketonization reactor, and the liquid crude absorber stream comprises greater than about 50 mole % of the acetone in the crude product mixture coming from the ketonization reactor, or the liquid crude absorber stream comprises greater than about 70 mole % of the acetone in the crude product mixture coming from the ketonization reactor, or the liquid crude absorber stream comprises greater than about 90 mole % of the acetone in the crude product mixture coming from the ketonization reactor.

The solvent-to-feed weight ratio is typically about 0.5:1 to about 3:1. The high heat of absorption of acetone, however, may require heat removal to minimize solvent flow, staging, and to enable the maximum recovery of acetone. For example, the heat of absorption may be removed by side draw coolers or by a heat-exchanged pump around loop in which liquid from the bottom effluent of the absorber is pumped through a heat exchanger and fed back into the column, typically about one-quarter to about one-half of the distance from the bottom of the column to the top. The flow in the pump around loop may be about 0.5 to about 10 times the flow of the crude acetone product removed from the bottom of the absorber, or about 1 to about 4 times the flow of the crude acetone product. For example, the temperature range of absorber operation can be about 10° to about 65° C., or about 25° to about 50° C. Any solvent with a suitable partition coefficient for acetone can be used in the absorber. Some representative examples of absorber solvents include, but are not limited to, water, C₅ to C₂₀ ketones, C₂ to C₁₆ carboxylic acids, C₆ to C₁₂ hydrocarbons, C₆ to C₁₆ ethers, C₅ to C₁₂ esters, and C₃ to C₁₂ alcohols. Some specific examples of absorber solvents are 2-pentanone, 4-methyl-2-pentaone, 2-heptanone, 5-methyl-2-hexanone, 4-heptanone, 2,4-dimethyl-5-pentanone, 2,5-dimethyl-4-heptanone, acetic acid, propionic acid, i-butyric acid, n-butyric acid, i-valeric acid, n-valeric acid, n-hexanoic acid, 2-ethyl-hexanoic acid, toluene, benzene, o-/m-/p-xylenes, diisopropyl ether, dipropylether, tertiary amyl methyl ether, dibutyl ether, n-propyl acetate, i-propyl acetate, n-butyl acetate, i-butyl acetate, sec-butyl acetate, 2-ethylhexyl acetate, ethyl propionate, n-propyl propionate, isopropyl propionate, ethyl n-butyrate, ethyl i-butyrate, methyl 2-ethylhexanoate, isopropanol, n-propanol, sec-butanol, i-butanol, n-butanol, n-hexanol, 2-ethylhexanol, and n-decanol.

With either condensation or absorption operations in the recovery zone, the carbon dioxide by-product stream will generally comprise about 95 to about 99.9 mole % carbon dioxide, 0 to about 0.4 mole % methane, 0 to about 0.5 mole % hydrogen, and about 0.02 to about 0.8 mole % isobutylene on an acetone and water free basis. Additionally, the carbon dioxide by-product stream may contain unrecovered acetone, typically 0.05 to 5 mole % acetone, water, 0.1 to 4 mole percent, and 0 to 100 ppm levels of other heavier by-products, based on the total weight of the carbon dioxide by-product stream.

The crude liquid acetone stream 75 obtained after condensation or absorption, i.e., the distillation feed, can comprise about 25 to about 70 weight % acetone, about 25 to about 75 weight % water, about 0.05 to about 2 weight % acetic acid, about 0.5 to about 2 weight % mesityl oxide and other related by-products such as, for example, isophorone and mesitylene, and from about 0.05 weight % to about 25 weight % of the impurity comprising at least one acetic acid azeotrope-forming compound, wherein the weight % is based on the total weight of the crude liquid acetone stream 75. The crude liquid acetone stream 75 can may also comprise about 25 to about 85 weight % acetone, about 15 to about 75 weight % water, about 0.05 to about 2 weight % acetic acid, about 0.5 to about 2 weight % mesityl oxide and other related by-products such as, for example, isophorone and mesitylene, and from about 0.1 weight % to about 20 weight % of the impurity comprising at least one acetic acid azeotrope-forming compound, based on the weight of the crude liquid acetone stream 75; or the crude liquid acetone stream 75 can comprise about 25 to about 95 weight % acetone, about 5 to about 75 weight % water, about 0.05 to about 2 weight % acetic acid, about 0.5 to about 2 weight % mesityl oxide and other related by-products such as, for example, isophorone and mesitylene, and from about 0.7 weight % to about 20 weight % of the impurity comprising at least one acetic acid azeotrope-forming compound. It is to be understood that the aforementioned weight percentages are based on the weight of the liquid crude acetone stream and the sum of the constituent weight percentages equals 100%.

This crude liquid acetone stream 75 can be fed to a distillation column where the liquid acetone stream from the recovery zone is separated into: i) a lower boiling fraction stream 82 comprising about 95 to about 99 weight % acetone, based on the weight of the constituents in the lower boiling fraction stream 82, and a minor amount of the water and the acetic acid azeotrope-forming compound; and ii) a higher boiling fraction stream 84 comprising a major amount of the water, the acetic acid azeotrope forming compound(s), and the ketonization byproducts.

The invention also includes having the crude liquid acetone stream separated into: i) a lower boiling fraction stream 82 comprising about 95 to about 99 weight % acetone, about 0.1 to about 5 weight % water and from about 50 ppm to about 1.0 weight % of the impurity having at least one acetic acid azeotrope-forming compound, based on the weight of the constituents in the lower boiling fraction stream 82; and ii) a higher boiling fraction stream 84 comprising greater than 50 weight % water, the remainder of the impurity and the ketonization byproducts, wherein the weight % is based on the weight of the constituents in the higher boiling fraction stream 84.

The invention also includes having the crude liquid acetone stream separated into: i) a lower boiling fraction stream 82 comprising about 95 to about 99 weight % acetone, about 0.1 to about 2 weight % water and from about 500 ppm to about 1 weight % of the impurity comprising at least one acetic acid azeotrope-forming compound, based on the weight of the constituents in the lower boiling fraction stream 82; and ii) a higher boiling fraction stream 84 comprising greater than 70 weight % water, the remainder of the impurity and the ketonization byproducts, wherein the weight % is based on the weight of the constituents in the higher boiling fraction 84.

The invention also includes after condensation or absorption and separation of the non-condensibles step from the crude product mixture, the crude acetone stream, or in the case where an absorber has been utilized, the crude liquid absorbent stream 75, is purified in a distillation zone 80 to produce a purified acetone stream 82 comprising at least 95 weight % acetone, based on the total weight of the acetone stream 82, and a minor amount of: water, the impurity comprising at least one azeotrope-forming compound, and ketonization byproducts present in the crude product mixture; a waste water stream 84, comprising water from the acid feed 15, any added steam 35, water created in the ketonization reactor, as well as any water added in the recovery zone 70; and a waste organic stream 86, comprising the azeotrope forming compounds, and the ketonization byproducts present in the recovered liquid acetone stream. Generally, waste water stream 84 is a collective stream from the various water sources comprising water from the acid feed 15, any added steam 35, water created in the ketonization reactor 60, as well as any water that may have been added in the recovery zone 70 via line 73. The waste organic stream 86 can also be a collective stream comprising by-product organics produced in the ketonization reactor 60, or non-ketonizable species brought in as impurities in the acid feed stream 15.

The invention also includes having the purified acetone stream 82 comprising from about 95 to about 99 weight % acetone, from about 0.1 to about 5 weight % water and from about 50 ppm to about 1.0 weight % of the impurity having at least one acetic acid azeotrope-forming compound, wherein the weight % is based on the total weight of the purified acetone stream 82.

The invention includes having the purified acetone stream 82 comprising from about 95 to about 99 weight % acetone, from about 0.1 to about 2 weight % water and from about 500 ppm to about 1 weight % of the impurity comprising at least one acetic acid azeotrope-forming compound, wherein the weight % is based on the total weight of the purified acetone stream 82.

As is recognized by those skilled in the art, the ranges specified herein include all numerical points between the beginning and ending delineated points, as has been expressly demonstrated herein.

The present invention is illustrated in greater detail by the specific examples presented below. It is to be understood that these examples are for illustrative purposes and are not intended to be limiting of the invention, but rather are to be construed broadly within the scope and content of the appended claims. All parts and percentages in the examples are on a weight basis unless otherwise stated.

### Examples 1-5

All analyses were carried out using gas chromatography (“GC”). Three GC methods had been developed and employed to analyze samples: GC Method-1 used a DB-Wax (60 m×0.32 mm×1.0 um) capillary column and a thermal conductivity detector (TCD). Samples were diluted in an internal standard solution and then injected into the GC. This method provided weight percent compositions of acetaldehyde, propionaldehyde, isobutyraldehyde, n-butyraldehyde, diethyl ether, acetone, water, isopropyl acetate, methyl ethyl ketone, isopropanol, isopropyl propionate, methyl propyl ketone, diethyl ketone, methyl isobutyl ketone, butyl acetate, mesityl oxide, dipropyl ketone, methyl amyl ketone, mesitylene, diacetone alcohol, and isophorone.

Due to the limitation for accurate determination of organic acids using the above direct injection GC Method-1, each sample was also analyzed by the following GC Method-2 that used a DB-1 (60 m×0.32 mm×1.0 um) capillary column and a flame ionization detector (FID). In this second GC method, the sample was first derivatized by reacting with BSTFA [N,O-bis(trimethylsilyl) trifluoroacetamide], which converted the organic acids into their corresponding trimethylsilyl (TMS) esters. These TMS esters are more volatile and inert for accurate quantification. Water also can be accurately quantified as its bis-TMS derivative when sufficient BSTFA reagent was applied. This method provided an accurate weight percentage of acetic acid, propionic acid, isobutyric acid, butyric acid, and formic acid, and method can also be used to quantify the weight percentage of alcohols (as TMS-ethers) and ketones (no derivatization reaction with BSTFA and detected as their original forms).

GC Method-3 was used to quantify PPM levels of impurities in the final distilled acetone samples. This method used multiple GC columns: DB-Wax (60 m×0.32 mm×1.0 um), DB-1 (60 m×0.32 mm×1.0 um), and DB-1301 (60 m×0.32 mm×1.0 um) with dual FID and a mass selective detector (GC/MS). Key aldehydes, alcohols, esters, and ketones at PPM concentration levels were quantified by the dual column GC/FID method and other impurities (ppm) were identified and estimated by GC/MS.

A typical ketonization procedure follows in which feedstocks for preparing acetone are various acetyl by-product mixtures containing azeotrope-forming impurities derived from:

Example 1—the acetylation of wood;

Examples 2 and 3—the preparation of specialty chemicals by acetylation;

Example 4—the preparation of isobutyric anhydride by the acetylation of isobutyric acid with acetic anhydride (TMCD process); and

Example 5—the production of diketene.

The acetyl by-product streams were mixed with sufficient water to hydrolyze any acetic anhydride present and to produce a wet acetic acid mixture. The composition of these streams are shown in Tables 1-5 in the columns labeled “Crude acetyl” and “Wet AcOH.” Any acetic anhydride present in the acetyl by-product streams was converted to acetic acid with the corresponding consumption of water. Small amounts of alpha-pinene and limonene were added to the hydrous acetic acid mixture from the acetylation of wood (Table 1) to bring the level of azeotrope-forming terpene impurities to easily detectable levels.

The ketonization reactor was operated continuously ranging from about 190 to about 506 hours. A 316 SS tubular ketonization reactor, ⅜″ ID×18″ L, with an ⅛″ ID thermowell was charged with a TiO2 (anatase)/4% graphite (as binder) catalyst that was ground and sieved to 10/20 mesh. Quartz chips were loaded above and below the catalyst bed. The reactor was placed in a clamshell furnace and connected via tubing to an evaporator unit comprising a 316 SS ½″ ID×12″ tube wrapped with a heating tape, a dual-barrel syringe pump and a level-controlled piston sludge pump. During operation of the reactor, the feed acid was pumped continuously to the evaporator at rates range from about 1.24 to about 1.52 g/min. The temperature of the evaporator was approximately 140° C. The vaporized wet acid stream was then passed to the ketonization reactor. The furnace heating element was adjusted to give a nominal inlet temperature to the catalyst zone of the reactor ranging from about 387° C. to about 415° C. throughout the run. The average temperature of the catalyst bed was about 399° C. to about 427° C. The outlet pressure of the condenser pot was atmospheric (about 730 torr).

The feed acid was sludged out of the evaporator at a rate of about 8 to about 30% of the feed flow. The reactor effluent was condensed at about 10° C., and allowed to collect in an insulated, cooled tank. The off gas from the tank was further cooled by dry ice and the condensed liquid collected in a trap. The volume of the off gas from the dry ice trap was measured by a flow meter and analyzed by gas chromatography. The material in the dry ice trap and product tank were weighed every 24 hours and analyzed by GC.

The composition and amount of the reactor feeds, sludge streams from the evaporator, and reactor effluent are given in Tables 1-6. All values are in weight percent, based on the total weights of the constituents in the stream, unless stated otherwise.

TABLE 1
(Example 1)

| | | | Sludge | | | | | |
|---|---|---|---|---|---|---|---|---|
| | Crude | Wet | from | Reactor | Reactor | | | |
| Component | Acetyl | AcOH | flash | Feed | Effluent | Distillate | | Bottoms |
| Acetic Acid | 95.7 | 89.5 | 94.7 | 88.3 | 0.3 | 0 | | 0.7 |
| Acetic Anhydride | 4.2 | ND | ND | ND | ND | 0 | | ND |
| Water | ND | 10.0 | 5.3 | 11.1 | 33.2 | 1.4 | | 96.4 |
| Acetone | ND | ND | ND | ND | 65.3 | 98.6 | | 0.1 |
| Mesityl Oxide | ND | ND | ND | ND | 0.3 | 1 | ppm | 0.8 |
| Impurities (total): | <0.1 | 0.5 | ND | 0.7 | 0.8 | 1346 | ppm | 2.1 |
| α-pinene | 125 ppm | 0.2 | ND | 0.3 | NS | ND | | ND |
| limonene | 134 ppm | 0.2 | ND | 0.3 | NS | ND | | ND |
| other terpenes | NS | 0.1 | ND | 0.1 | NS | 346 | ppm | ND |

NS = not speciated.

TABLE 2
(Example 2)

| | | | Sludge | | | | |
|---|---|---|---|---|---|---|---|
| | Crude | Wet | from | Reactor | Reactor | | |
| Component | Acetyl | AcOH | flash | Feed | Effluent | Distillate | Bottoms |
| Acetic Acid | 92.7 | 84.1 | 69.1 | 84.1 | 0.0 | ND | 0.5 |
| Acetic Anhydride | ND | ND | ND | ND | ND | ND | ND |
| Water | ND | 9.3 | 2.3 | 10.0 | 34.2 | 0.5 | 78.9 |
| Acetone | ND | ND | ND | ND | 58.6 | 99.5 | 2.5 |
| Mesityl Oxide | ND | ND | ND | ND | 0.1 | 1 ppm | 0.2 |
| Impurities (total): | 7.3 | 6.6 | 28.6 | 0.7 | 4.6 | 62 ppm | 17.9 |
| isobutylbenzene | 3.6 | 3.2 | ND | 3.5 | 3.5 | ND | 13.7 |
| other alkylbenzenes | | | | | | | |
| isobutylacetophenone | 1.1 | 1.0 | ND | 1.1 | 1.1 | ND | 4.2 |
| other acetophenones | | | | | | | |
| heavy aromatics and | 2.6 | 2.4 | 28.6 | NS | NS | ND | NS |
| ketones | | | | | | | |
| HF and other F | 2.6 ppm | NS | ND | 2.5 ppm | 3 ppm | ND | 8.3 ppm |
| compounds | | | | | | | |

TABLE 3
(Example 3)

| | | | Sludge | | | | |
|---|---|---|---|---|---|---|---|
| | Crude | Wet | from | Reactor | Reactor | | |
| Component | Acetyl | AcOH | flash | Feed | Effluent | Distillate | Bottoms |
| Acetic acid | 98.3 | 90.0 | 94.1 | 89.4 | 0.1 | ND | 0.3 |
| Acetic anhydride | 1.6 | ND | ND | ND | ND | ND | ND |
| Water | ND | 9.9 | 5.9 | 10.4 | 34.5 | 0.7 | 98.6 |
| Acetone | ND | ND | ND | ND | 65.1 | 99.3 | 0.1 |
| Mesityl Oxide | ND | ND | ND | ND | 0.2 | 1 ppm | 0.5 |
| Impurities (total): | 0.2 | 0.2 | 0 | 0.2 | 0.1 | 180 ppm | 0.4 |
| phenylacetates | 0.2 | 0.2 | NS | 0.2 | 0.1 | ND | 0.4 |

TABLE 4
(Example 4)

| | | | Sludge | | | | |
|---|---|---|---|---|---|---|---|
| | Crude | Wet | from | Reactor | Reactor | | |
| Component | Acetyl | AcOH | flash | Feed | Effluent | Distillate | Bottoms |
| Acetic acid | 98.8 | 88.2 | 94.2 | 87.2 | 1.1 | ND | 2.6 |
| Acetic anhydride | <0.1 | ND | ND | ND | ND | ND | ND |
| Water | ND | 10.7 | 5.4 | 11.5 | 38.6 | 0.3 | 92.9 |
| Acetone | <0.1 | <0.1 | <0.1 | <0.1 | 58.5 | 99.4 | 0.5 |
| Mesityl Oxide | ND | ND | ND | ND | 0.4 | 18 ppm | 0.5 |
| Impurities (total): | 1.2 | 1.1 | 0.4 | 1.2 | 1.4 | 0.7 | 3.6 |
| Tetramethylethylene | 0.1 | 0.1 | NS | NS | 0.1 | 0.03 | NS |
| 2,4-Dimethyl-1,3- | 0.1 | 0.1 | NS | NS | NS | 5 ppm | NS |
| pentadiene and | | | | | | | |
| isomers | | | | | | | |
| Diisopropyl ketone | 0.2 | 0.2 | NS | NS | NS | 23 ppm | NS |
| Isopropyl isopropenyl | 0.7 | 0.6 | NS | NS | NS | ND | NS |
| ketone | | | | | | | |
| Others | 0.1 | 0.1 | .4 | 1.2 | 1.3 | <0.1 | 3.6 |

TABLE 5
(Example 5)

| | | | Sludge | | | | |
|---|---|---|---|---|---|---|---|
| | Crude | Wet | from | Reactor | Reactor | | |
| Component | Acetyl | AcOH† | flash | Feed | Effluent | Distillate | Bottoms |
| Acetic acid | 58.9 | 85.0 | 89.0 | 83.8 | 0.7 | ND | 1.8 |
| Acetic anhydride | ND | ND | ND | ND | ND | ND | ND |
| Water | 26.0 | 8.3 | 7.1 | 8.7 | 32.9 | 0.4 | 93.1 |
| Acetone | 11.5 | 5.4 | 1.9 | 6.5 | 64.1 | 99.6 | 1.7 |
| Mesityl Oxide | ND | ND | ND | ND | 0.7 | 5.5 ppm | 1.3 |
| Impurities (total): | 3.6 | 1.3 | 2.1 | 1.1 | 1.6 | 82 ppm | 1.9 |
| Isophorone | ND | ND | NS | ND | 0.1 | ND | 0.1 |
| Others* | 3.6 | 1.3 | 2.1 | 1.1 | 1.5 | ND | 1.8 |

*includes diketene, 2,4-pentanedione, isopropenyl acetate, 2,4,6-heptatrione, 2,6-dimethylpyrone, and dehydroacetic acid

TABLE 6

| | | | Feed | | | Avg. | |
|---|---|---|---|---|---|---|---|
| | | Feed | to | Feed | | Cat. | |
| | | Flow | Flash | to | Feed | Bed | Reactor |
| | Cat | Rate | Unit | Reactor | Temp. | Temp. | Effluent |
| Stream | (g) | (g/min) | (g) | (g) | (° C.) | (° C.) | (g) |
| Ex. 1 | 11.2 | 1.25 | 37,465 | 30,512 | 410-415 | 410-413 | 21,505 |
| Ex. 2 | 21.0 | 1.52 | 18,040 | 16,684 | 387-402 | 399-407 | 10,419 |
| Ex. 3 | 20.0 | 1.24 | 17,392 | 15,202 | 407 | 425 | 10,258 |
| Ex. 4 | 15.0 | 1.24 | 37,344 | 32,746 | 401 | 427 | 21,470 |
| Ex. 5 | 15.5 | 1.2 | 28,093 | 21,493 | 400-418 | 427 | 14,027 |

The crude acetone product from the ketonization reactor was fed to a 1 in (ID)×8 ft, 316 stainless steel distillation column packed with ⅛ in cannon (Penn State), 316 stainless steel packing. The column was equipped with a reflux pot and piston pump to feed condensate back to the column as reflux. The overhead vapors from the column were condensed into a reflux pot. A portion of the condensate in the reflux pot was pumped back into the top of the column to maintain a reflux ratio of about 1.0 to about 1.5 using a piston pump. Overflow from the reflux pot was collected as distillate. The reboiler was a 250 mL stainless steel vessel. Heat was supplied to the reboiler using a 150-watt band heater. The pot level of the reboiler was controlled via takeoff with a piston pump. The pressure at the top of the column was about 725-730 torr at a temperature of 54-55° C. The temperature at the bottom of the column was about 99-107° C. The crude acetone product was fed continuously at a rate of about 5.4 mL/min to the 4 foot point of column using a piston pump. The compositions of the distillate and distillate bottoms are shown in Tables 1-5. The total weights of the crude acetone fed to the column, the distillate, and distillate bottoms are shown in Table 6.

The composition of the CO₂ offgas from the ketonization reactor is shown in Table 7. The various azeotrope forming impurities present in the acetyl byproduct streams, their compositions, and boiling point data are given in Table 8.

TABLE 7

| Stream | CO2 | Methane | Hydrogen | Isobutylene |
|---|---|---|---|---|
| Ex. 1 | 99.77 | 0.03 | 0.04 | 0.15 |
| Ex. 2 | 99.78 | 0.00 | 0.05 | 0.17 |
| Ex. 3 | 99.83 | 0.08 | 0.08 | 0.10 |
| Ex. 4 | 99.78 | 0.08 | 0.08 | 0.10 |
| Ex. 5 | 99.47 | 0.32 | 0.04 | 0.17 |

TABLE 8

| | | | BP | BP | BP | Weight |
|---|---|---|---|---|---|---|
| | Azeo | Azeo | Comp. 1 | Comp. 2 | Azeotrope | percent |
| Source | Comp. 1 | Comp. 2 | (° C.) | (° C.) | (760 torr) | Comp. 1 |
| All | Acetone | isophorone | 56.1 | 215.2 | No | No |
| feeds | | | | | azeotrope | azeotrope |
| All | Acetone | mesityl | 56.1 | 129.5 | No | No |
| feeds | | oxide | | | azeotrope | azeotrope |
| All | Acetone | mesitylene | 56.1 | 164.6 | No | No |
| feeds | | | | | azeotrope | azeotrope |
| All | Acetic | isophorone | 118 | 215.2 | No | No |
| feeds | Acid | | | | azeotrope | azeotrope |
| All | Acetic | mesityl | 118 | 129.5 | No | No |
| feeds | Acid | oxide | | | azeotrope | azeotrope |
| All | Acetic | mesitylene | 118 | 164.6 | No | No |
| feeds | Acid | | | | azeotrope | azeotrope |
| All | water | isophorone | 100 | 215.2 | 99.5 | 83.90% |
| feeds | | | | | | |
| All | water | mesityl | 100 | 129.5 | 91.8 | 34.80% |
| feeds | | oxide | | | | |
| All | water | mesitylene | 100 | 164.6 | 96.5 | not |
| feeds | | | | | | reported |
| Ex. 1 | Acetone | α-pinene | 56.1 | 155.8 | No | No |
| | | | | | azeotrope | azeotrope |
| Ex. 1 | Acetone | Fenchone | 56.1 | 193.0 | No | No |
| | | | | | azeotrope | azeotrope |
| Ex. 1 | Acetone | isobornyl | 56.1 | 225.8 | No | No |
| | | acetate | | | azeotrope | azeotrope |
| Ex. 1 | Acetone | limonene | 56.1 | 177.7 | No | No |
| | | | | | azeotrope | azeotrope |
| Ex. 1 | Acetic | α-pinene | 118 | 155.8 | 113.4 | 69.4 |
| | Acid | | | | | |
| Ex. 1 | Acetic | Fenchone | 118 | 193.0 | No | No |
| | Acid | | | | azeotrope | azeotrope |
| Ex. 1 | Acetic | isobornyl | 118 | 225.8 | No | No |
| | Acid | acetate | | | azeotrope | azeotrope |
| Ex. 1 | Acetic | limonene | 118 | 177.7 | 115.8 | 82.8 |
| | Acid | | | | | |
| Ex. 1 | water | isobutylbenzene | 100 | 172.8 | 97.7 | 60.3 |
| Ex. 1 | water | limonene | 100 | 177.18 | 98.3 | 63.3 |
| Ex. 1 | water | α-pinene | 100 | 156.45 | 95.5 | 43.0 |
| Ex. 2 | Acetone | isobutylbenzene | 56.1 | 172.8 | No | No |
| | | | | | azeotrope | azeotrope |
| Ex. 2 | Acetone | isobutyl- | 56.1 | 267 | No | No |
| | | acetophenone | | | azeotrope | azeotrope |
| Ex. 2 | Acetic | isobutyl- | 118 | 267 | No | No |
| | Acid | acetophenone | | | azeotrope | azeotrope |
| Ex. 2 | Acetic | isobutylbenzene | 118 | 172.8 | pinched | pinched |
| | Acid | | | | | |
| Ex. 2 | water | isobutyl- | 100 | 267 | 99.8 | 92.30% |
| | | acetophenone | | | | |
| Ex. 2 | water | isobutylbenzene | 100 | 172.8 | 97.7 | 60.30% |
| Ex. 3 | Acetone | phenylacetate | 56.1 | 195.7 | No | No |
| | | | | | azeotrope | azeotrope |
| Ex. 3 | Acetic | phenylacetate | 118 | 195.7 | No | No |
| | Acid | | | | azeotrope | azeotrope |
| Ex. 3 | water | phenylacetate | 100 | 195.7 | 99.3 | 83.60% |
| Ex. 4 | Acetone | 2,4- | 56.1 | 93.3 | No | No |
| | | dimethyl-1,3- | | | azeotrope | azeotrope |
| | | pentadiene | | | | |
| Ex. 4 | Acetone | DIPK | 56.1 | 124.4 | No | No |
| | | | | | azeotrope | azeotrope |
| Ex. 4 | Acetone | isopropyl | 56.1 | 130 | No | No |
| | | isopropenyl | | | azeotrope | azeotrope |
| | | ketone | | | | |
| Ex. 4 | Acetone | tetramethyl- | 56.1 | 73.2 | 53.5 | 65.10% |
| | | ethylene | | | | |
| Ex. 4 | Acetic | 2,4- | 118 | 93.3 | 83.9 | 18% |
| | Acid | dimethyl-1,3- | | | | |
| | | pentadiene | | | | |
| Ex. 4 | Acetic | DIPK | 118 | 124.4 | Pinched | Pinched |
| | Acid | | | | | |
| Ex. 4 | Acetic | isopropyl | 118 | 130 | 117.9 | 93.70% |
| | Acid | isopropenyl | | | | |
| | | ketone | | | | |
| Ex. 4 | Acetic | tetramethyl- | 118 | 73.2 | Pinched | Pinched |
| | Acid | ethylene | | | | |
| Ex. 4 | water | 2,4- | 100 | 93.3 | 76.8 | 13.00% |
| | | dimethyl- | | | | |
| | | 1,3- | | | | |
| | | pentadiene | | | | |
| Ex. 4 | water | DIPK | 100 | 124.4 | 92.9 | 34.00% |
| Ex. 4 | water | isopropyl | 100 | 130 | 92.9 | 33.00% |
| | | isopropenyl | | | | |
| | | ketone | | | | |
| Ex. 4 | water | tetramethyl- | 100 | 73.2 | 65.8 | 6.70% |
| | | ethylene | | | | |
| Ex. 5 | Acetone | 2,4- | 56.1 | 137 | No | No |
| | | pentanedione | | | azeotrope | azeotrope |
| Ex. 5 | Acetone | isopropenyl | 56.1 | 96.9 | No | No |
| | | acetate | | | azeotrope | azeotrope |
| Ex. 5 | Acetic | 2,4- | 118 | 137 | Pinched | Pinched |
| | Acid | pentanedione | | | | |
| Ex. 5 | Acetic | isopropenyl | 118 | 96.9 | No | No |
| | Acid | acetate | | | azeotrope | azeotrope |
| Ex. 5 | water | 2,4- | 100 | 137 | 96.1 | 45.00% |
| | | pentanedione | | | | |
| Ex. 5 | water | isopropenyl | 100 | 96.9 | 81.6 | 16.30% |
| | | acetate | | | | |

### Comparative Example 6

The following prophetic example illustrates the difficulty in attempting purification of acetic acid containing impurities that form azeotropes or are pinched using distillative processes. The continuous distillations of eight mixtures of acetic acid with azeotrope-forming or pinch-forming impurities were simulated to give predicted distillate and bottoms compositions. The compositions of these mixtures are shown in Table 9. Each feed mixture was 95/5 weight ratio of acetic acid to impurities. The distillation column contained twelve theoretical stages. The reflux ratio was around 2, except for cases with extremely difficult separation, where it was varied to improve the separation.

TABLE 9

| | | System | # Theor. | Reflux | Feed | HOAc | Impuriy |
|---|---|---|---|---|---|---|---|
| Impurity | Source | Type | Stages | Ratio | kg | (Wt. %) | (Wt. %) |
| limonene | Ex. 1 | azeotrope | 12 | 1.5 | 100 | 95% | 5% |
| alpha pinene | Ex. 1 | azeotrope | 12 | 2.5 | 100 | 95% | 5% |
| isobutylbenzene | Ex. 2 | Pinched | 12 | 4 | 100 | 95% | 5% |
| 2,4-dimethyl-1,3- | Ex. 4 | Azeotrope | 12 | 4.6 | 100 | 95% | 5% |
| pentadiene | | | | | | | |
| tetramethylethylene | Ex. 4 | Pinched | 12 | 1.9 | 100 | 95% | 5% |
| diisopropyl ketone | Ex. 4 | Pinched | 12 | 9.9 | 100 | 95% | 5% |
| isopropyl isopropenyl | Ex. 4 | azeotrope | 12 | 14 | 100 | 95% | 5% |
| ketone | | | | | | | |
| 2,4-pentanedione | Ex. 5 | Pinched | 12 | 2 | 100 | 95% | 5% |

AcOH is acetic acid.

Distillate and bottoms compositions from the simulation are summarized in Table 10.

TABLE 10

| | | Distillate | HOAc | Impurity | Bottoms | HOAc | Impurity |
|---|---|---|---|---|---|---|---|
| Impurity | Source | kg | (Wt. %) | (Wt. %) | kg | (Wt. %) | (Wt. %) |
| limonene | Ex. 1 | 27.3 | 83.0% | 17.0% | 72.7 | 99.50% | 0.5% |
| alpha pinene | Ex. 1 | 15.1 | 69.8% | 30.3% | 84.9 | 99.50% | 0.5% |
| isobutylbenzene | Ex. 2 | 59.4 | 96.7% | 3.3% | 40.6 | 92.51% | 7.5% |
| 2,4-dimethyl-1,3- | Ex. 4 | 5.6 | 18.8% | 81.2% | 94.4 | 99.50% | 0.5% |
| pentadiene | | | | | | | |
| tetramethylethylene | Ex. 4 | 4.8 | 9.7% | 90.3% | 95.2 | 99.27% | 0.7% |
| DIPK | Ex. 4 | 86.5 | 95.4% | 4.6% | 13.5 | 92.64% | 7.4% |
| isopropyl isopropenyl | Ex. 4 | 57.6 | 94.4% | 5.6% | 42.4 | 95.78% | 4.2% |
| ketone | | | | | | | |
| 2,4-pentanedione | Ex. 5 | 88.4 | 99.6% | 0.4% | 11.6 | 59.57% | 40.4% |

Acetic acid recovery and percent removal of impurity are presented in Table 11. Note that in all cases high recovery and high levels of impurity removal could not be achieved simultaneously.

TABLE 11

| | | Stream w/ | | |
|---|---|---|---|---|
| | | Highest Purity | % Recov | % Removal |
| Impurity | Source | Acetic Acid | of AcOH | of Impurity |
| limonene | Ex. 1 | bottoms | 76% | 93% |
| alpha pinene | Ex. 1 | bottoms | 89% | 92% |
| isobutylbenzene | Ex. 2 | distillate | 40% | 61% |
| 2,4-dimethyl-1,3- | Ex. 4 | bottoms | 99% | 91% |
| pentadiene | | | | |
| tetramethylethylene | Ex. 4 | bottoms | 99.5% | 86% |
| DIPK | Ex. 4 | distillate | 87% | 20% |
| isopropyl | Ex. 4 | distillate | 57% | 36% |
| isopropenyl ketone | | | | |
| 2,4-pentanedione | Ex. 5 | distillate | 93% | 94% |

Having described the invention in detail, those skilled in the art will appreciate that modifications may be made to the various examples of the invention without departing from the scope and spirit of the invention disclosed and described herein. It is, therefore, not intended that the scope of the invention be limited to the specific examples illustrated and described but rather it is intended that the scope of the present invention be determined by the appended claims and their equivalents.

What is claimed is:

1. A process for preparing a ketone from an acetic acid containing stream, comprising:
a. contacting an acetyl feed stream comprising:
i) acetic acid; and
ii) an impurity comprising at least one acetic acid azeotrope-forming compound,
with a metal oxide catalyst in a ketonization reactor to produce a crude product mixture comprising acetone, water, said impurity, and ketonization byproducts.

2. The process of claim 1 further comprising:
b. distilling the crude product mixture to recover:
i) a lower boiling fraction comprising acetone, and a minor amount of: water, the impurity, and the ketonization byproducts; and
ii) a higher boiling fraction comprising a major amount of: water, the impurity, and the ketonization byproducts.

3. A process for preparing a ketone from an acetic acid containing stream, comprising:
a. contacting an acetyl feed stream comprising:
i) acetic acid; and
ii) an impurity comprising at least one acetic acid azeotrope-forming compound,
with a metal oxide catalyst in a ketonization reactor to produce a crude product mixture comprising acetone, water, said impurity, and ketonization byproducts; and
b. distilling the crude product mixture to recover:
i) a lower boiling fraction stream comprising acetone, and a minor amount of: water, the impurity, and the ketonization byproducts; and
ii) a higher boiling fraction stream comprising a major amount of: water, the impurity, and the ketonization byproducts.

4. The process according to claim 1, further comprising mixing said acetyl feed stream with water prior to step (a), wherein the feed stream comprises about 40 to about 99 weight % acetic acid, up to about 50 weight of the impurity, and optionally up to about 30 weight % water, wherein the weight % is based on the total weight of the feed stream.

5. The process according to claim 4, wherein the acetic acid is produced from a process for the acetylation of a compound selected from the group consisting of an alcohol, a polyol, cellulose, an amine, carboxylic acid, and an aromatic compound by contacting said compound with acetic anhydride.

6. The process according to claim 5 wherein the acetic acid is recovered from a process selected from the group consisting of wood acetylation; acetylation of alcohols with acetic anhydride to form esters; carbonylation of methanol and methyl acetate to form acetic acid and acetic anhydride; preparation of 2,2,4,4-tetramethyl-1,3-cyclobutanediol from isobutyric anhydride via acetic anhydride exchange; preparation of ketenes and diketene from acetic acid; condensation of phenyl acetate monomers; preparation of fine chemicals and pharmaceuticals; preparation of carboxylic acid anhydrides from their corresponding acids by exchange with acetic anhydride; acylation reactions and mixtures thereof.

7. The process according to claim 1 wherein the impurity is selected from the group consisting of an alkyl aromatic hydrocarbon, a ketone, an aromatic ester, an acyclic ester, a terpene, a terpenoid, an acyclic unsaturated hydrocarbon, and combinations thereof.

8. The process according to claim 1, wherein the feed stream is contacted with said catalyst at a temperature of from about 350° to about 650° C. and a pressure of from about 10 kPa to about 3000 kPa and the catalyst comprises a metal oxide selected from the group consisting of titanium, zirconium, thorium, cerium, lanthanum, magnesium, aluminum, and mixtures thereof.

9. The process according to claim 1 wherein said metal oxide catalyst further comprises from about 1 weight % to about 25 weight %, based on the total weight of the catalyst, of lithium, sodium, potassium, cesium, or mixtures thereof.

10. The process according to claim 1 wherein the crude product mixture comprises a liquid crude acetone stream comprising about 25 to about 70 weight % acetone, about 25 to about 75 weight % water, and about 0.05 weight % to about 25 weight % of said impurity, wherein the weight % is based on the weight of the liquid crude acetone stream.

11. The process according to claim 1, further comprising contacting the crude product mixture with water in a countercurrent absorber prior to said distilling step (b).

12. The process according to claim 2 wherein the lower boiling fraction comprises from about 95 to about 99 weight % acetone and from about 50 ppm to about 1 weight % of said impurity, wherein the weight % is based on the total weight of the lower boiling fraction stream.

13. The process according to claim 12, wherein the higher boiling fraction stream comprises greater than 50 weight % water, and the remainder of the impurity and ketonization byproducts, wherein the weight % is based on the total weight of the higher boiling fraction.

14. A process for preparing a ketone from an acetic acid containing stream, comprising:
a. contacting an acetyl feed stream comprising:
i) acetic acid; and
ii) an impurity comprising at least one acetic acid azeotrope-forming compound,
with a metal oxide catalyst in a ketonization reactor to produce a crude product mixture comprising acetone, water, the impurity, and ketonization byproducts;
b. contacting the crude product mixture with an absorption solvent to produce a liquid crude absorbent stream having greater than 50 mole % of the acetone in the crude product mixture and a gaseous crude absorbent stream comprising carbon dioxide and less than 50 mole % of the acetone in the crude product mixture; and
c. distilling the crude liquid absorbent stream to recover:
i) a lower-boiling fraction stream comprising at least about 95 weight % acetone and a minor amount of: water, the impurity, and ketonization by-products present in the crude product mixture, wherein the weight % is based on the total weight of the lower-boiling fraction stream; and
ii) a higher boiling fraction comprising a major amount of: water, the impurity, and the ketonization by-products present in the crude product mixture.

15. The process according to claim 14, wherein the acetic acid is produced from a process for the acetylation of a compound selected from the group consisting of an alcohol, a polyol, cellulose, an amine, carboxylic acid, and an aromatic compound by contacting said compound with acetic anhydride.

16. The process according to claim 15 wherein the acetic acid is recovered from a process selected from the group consisting of wood acetylation; acetylation of alcohols with acetic anhydride to form esters; carbonylation of methanol and methyl acetate to form acetic acid and acetic anhydride; preparation of 2,2,4,4-tetramethyl-1,3-cyclobutanediol from isobutyric anhydride via acetic anhydride exchange; preparation of ketenes and diketene from acetic acid; condensation of phenyl acetate monomers; preparation of fine chemicals and pharmaceuticals; preparation of carboxylic acid anhydrides from their corresponding acids by exchange with acetic anhydride; acylation reactions and mixtures thereof.

17. The process according to claim 15 wherein the impurity is selected from the group consisting of an alkyl aromatic hydrocarbon, a ketone, an aromatic ester, an acyclic ester, a terpene, a terpenoid, an acyclic unsaturated hydrocarbon, and combinations thereof.

18. The process according to claim 17 wherein the ketonization catalyst comprises one or more metal oxides of titanium, zirconium, lanthanum, cerium, thorium, or mixtures and includes from about 1 weight % to about 25 weight %, based on the total weight of the catalyst, of lithium, sodium, potassium, cesium, or a mixture thereof, and has a surface area of about 10 to about 400 m²/g of catalyst.

19. The process according to claim 17 wherein said contacting step is carried out in a adiabatic fixed bed reactor having a temperature of about 300 to about 600° C. over the length of the reactor and an inlet pressure of about 0.5 to about 10 bars absolute.

20. The process of claim 14 wherein the liquid crude acetone stream comprises about 25 to about 70 weight % acetone, about 25 to about 75 weight % water, and about 0.05 weight % to about 25 weight % of said impurity, based on the total weight of the crude acetone stream.

21. The process of claim 14 wherein said lower boiling fraction stream comprises about 95 to about 99 weight % acetone, about 0.1 to about 5 weight % water and from about 50 ppm to about 1.0 weight % of the impurity having at least one acetic acid azeotrope-forming compound, wherein the weight % is based on the total weight of the lower boiling fraction stream; and the higher boiling fraction stream comprising greater than 50 weight % water, the remainder of the acetic acid azeotrope forming compound(s) and the ketonization byproducts, wherein the weight % is based on the total weight of the higher boiling fraction stream.

22. The process of claim 14 wherein said lower boiling fraction stream comprises about 95 to about 99 weight % acetone, about 0.1 to about 2 weight % water and from about 500 ppm to about 1 weight % of the impurity comprising at least one acetic acid azeotrope-forming compound, wherein the weight % is based on the total weight of the lower boiling fraction stream; and the higher boiling fraction stream comprising greater than 70 weight % water, the remainder of the acetic acid azeotrope forming compound(s) and the ketonization byproducts, wherein the weight % is based on the total weight of the higher boiling fraction stream.

23. A process for preparing a ketone from an acetic acid containing stream, comprising the steps of:
a) vaporizing an acetyl feed stream comprising acetic acid, an impurity comprising at least one acetic acid azeotrope-forming compound, and 0-50 weight % water, based in the total weight of the acetyl feed stream;
b) optionally mixing steam with the vaporized acetyl feed stream to produce a vaporized feed mixture;
c) superheating said vaporized feed mixture to produce a superheated feed mixture;
d) contacting said superheated feed mixture with a metal oxide catalyst in a ketonization reactor to produce a crude product mixture from a ketonization reaction comprising acetone, water, the impurity, carbon dioxide, and ketonization byproducts;
e) recovering acetone from condensable components of the crude product mixture to produce a liquid crude acetone stream and a gaseous off-gas stream;
e) distilling the liquid crude acetone stream to recover:
i) a purified acetone stream comprising at least 95 weight % acetone and a minor amount of: water, the azeotrope-forming compound, and ketonization byproducts, wherein the weight % is based on the total weight of the purified acetone stream;
ii) a waste water stream comprising a major amount of water in the recovered liquid acetone stream, and;
iii) a waste organic stream comprising the azeotrope forming compounds, and the ketonization byproducts present in the recovered liquid acetone stream.

24. The process of claim 23 wherein liquid crude acetone stream comprising about 25 to about 70 weight % acetone, about 25 to about 75 weight % water, and about 0.05 weight % to about 25 weight % of said impurity, wherein the weight % is based on the weight of the liquid crude acetone stream.

25. The process of claim 23 wherein the purified acetone stream comprises from about 95 to about 99 weight % acetone, from about 0.1 to about 5 weight % water and from about 50 ppm to about 1.0 weight % of the impurity having at least one acetic acid azeotrope-forming compound, wherein the weight % is based on the total weight of the purified acetone stream.

26. The process of claim 23 wherein the purified acetone comprises from about 95 to about 99 weight % acetone, from about 0.1 to about 2 weight % water and from about 500 ppm to about 1 weight % of the impurity comprising at least one acetic acid azeotrope-forming compound, wherein the weight % is based on the total weight of the purified acetone stream.
